(19) United States
(12) Patent Application Publication
(10) Pub. No.: US 2017/0049453 A1
(43) Pub. Date: Feb. 23, 2017

(54) MULTIPLE OCCLUDER DELIVERY DEVICES AND METHODS

(71) Applicant: Amsel Medical Corporation, Cambridge, MA (US)

(72) Inventors: Arnold Miller, Cambridge, MA (US); Raanan A. Miller, Cambridge, MA (US); Nir Lilach, Kfar Yehoshua (IL)

(21) Appl. No.: 15/226,577

(22) Filed: Aug. 2, 2016

Related U.S. Application Data
(63) Continuation-in-part of application No. 14/639,814, filed on Mar. 5, 2015.
Application No. 14/639,814 is a continuation-in-part of application No. 14/272,304, filed on May 7, 2014.
Application No. 14/272,304 is a continuation-in-part of application No. 13/857,424, filed on Apr. 5, 2013.
Application No. 13/857,424 is a continuation-in-part of application No. 13/348,416, filed on Jan. 11, 2012.
(60) Provisional application No. 62/203,655, filed on Aug. 11, 2015.
(60) Provisional application No. 62/298,724, filed on Feb. 23, 2016.
(60) Provisional application No. 62/084,989, filed on Nov. 26, 2014.
(60) Provisional application No. 61/948,241, filed on Mar. 5, 2014.
(60) Provisional application No. 61/820,589, filed on May 7, 2013.
(60) Provisional application No. 61/620,787, filed on Apr. 5, 2012.
(60) Provisional application No. 61/431,609, filed on Jan. 11, 2011.

(51) Int. Cl. A61B 17/128; A61B 17/122

(52) U.S. Cl. CPC A61B 17/1285; A61B 17/122; A61B 2017/00778

(57) ABSTRACT

The present invention provides a device for delivering multiple occluders laparoscopically, percutaneously, and for open surgical procedures. Occluders can be delivered in a minimally invasive procedure to a hollow structure such as a blood vessel. The device allows occlusion without requiring catheterization or extraction of the needle from the body between occluder deployments. An occluder delivery device similar to a syringe is provided that injects occluders directly to the region of interest. The device can hold several occluders at a time, so that multiple occluders can be delivered to a desired location or to multiple nearby locations with only a single injection of the delivery needle.

### CROSS-REFERENCE TO RELATED APPLICATIONS

This application claims the benefit of and priority to U.S. Provisional Application Ser. No. 62/203,655, filed Aug. 11, 2015, and U.S. Provisional Application Ser. No. 62/298,724, filed Feb. 23, 2016, and is a continuation-in-part of U.S. application Ser. No. 14/639,814, filed Mar. 5, 2015 (which claims the benefit of and priority to U.S. Provisional Application Ser. No. 62/084,989, filed Nov. 26, 2014), which is a continuation-in-part of U.S. application Ser. No. 14/272,304, filed May 7, 2014 (which claims the benefit of and priority to U.S. Provisional Application Ser. No. 61/948,241, filed Mar. 5, 2014, and U.S. Provisional Application Ser. No. 61/820,589, filed May 7, 2013), which is a continuation-in-part of U.S. application Ser. No. 13/857,424, filed Apr. 5, 2013 (which claims the benefit of and priority to U.S. Provisional Application Ser. No. 61/620,787, filed Apr. 5, 2012), which is a continuation-in-part of U.S. application Ser. No. 13/348,416, filed Jan. 11, 2012 (which claims the benefit of and priority to U.S. Provisional Application Ser. No. 61/431,609, filed Jan. 11, 2011), the entire contents of each of which are incorporated by reference herein in their entirety.

### FIELD OF THE INVENTION

The invention generally relates to the occlusion of hollow structures such as blood vessels, and more specifically to percutaneous delivery of occluders.

### BACKGROUND

Varicoceles, varicose veins, and ducts such as the cystic duct, or the left atrial appendage, are all examples of hollow structures that under certain circumstances benefit from being securely occluded with minimally invasive procedures. Varicocele is one of the leading causes of male infertility. It is characterized by an enlargement of the pampiniform venous plexus, which is a group of veins that drains blood from the testicles. Defective valves or compression of the veins can lead to the formation of a varicocele.

One known method of treatment involves inserting a catheter through a vein in the thigh, guiding it through the vasculature to the affected area, and closing off the malfunctioning veins. Closing off of veins may involve injecting a fluid or inserting a coil or other apparatus to block the blood flow through the veins. But those methods suffer from certain complications. The catheterization procedure may last one or two hours, and there is risk of hematoma, hydrocele, or other injury to the scrotum or other tissues. The artery that supplies blood to the testicle may be injured by these procedures as well.

### SUMMARY

The present invention provides a device for delivering multiple occluders laparoscopically, thoracoscopically, percutaneously, and for open surgical procedures. Occluders can be delivered in a minimally invasive procedure to a hollow structure such as a blood vessel. The device allows occlusion of such structures without requiring catheterization. An occluder delivery device similar to a syringe is provided that injects occluders directly to the region of interest. The device can hold several occluders at a time, so that multiple occluders can be delivered to a desired location or to multiple nearby locations with only a single injection of the delivery needle or a single insertion of the laparoscopic device. The multiple occluders can also be used in some applications to attach, or fix multiple tissue and/or material layers together, or can be used as a marker or indicator for follow on surgical procedures.

The multiple-occluder delivery device is useful for treatment of varicocele, among other conditions, where the region to be mechanically occluded is larger than the closure region of any single occluder element. It is useful for any procedure where a hollow structure, cavity, duct, or tubular structure is to be occluded. It is also useful for connecting two structures together. To treat varicocele, for example, the delivery device is inserted and the tube containing the occluders is pushed through the target blood vessel. The distal-most occluder is pushed out of the needle, where it expands on the far side of the vessel. The device is pulled back through the vessel, leaving the distal occluder transfixing the vessel. A proximal occluder is pushed out of the needle, and the two occluder elements are pushed towards each other until they lock together. Additional occluders that are lined up in the delivery needle can be deployed in a similar manner without having to withdraw the delivery device from the patient. The multiple-occluder delivery device thus allows several occluders to be implanted with only a single insertion of the needle.

In certain aspects, the invention provides a device for percutaneously or laparoscopically delivering at least one, but typically multiple occluders to a hollow structure. The device includes a hollow tube having a proximal end and a distal end. Several occluders are disposed serially within the tube, each occluder comprising a distal occluder element and a proximal occluder element. The device further comprises a pushing member configured to push an occluder out of the distal end of the tube.

In embodiments, the distal occluder element and the proximal occluder element are separate from but connectable to each other. The distal occluder element may comprise a connecting rod comprising a first connection element. The proximal occluder element may comprise a second connection element. When the distal and proximal occluder elements are pushed together, the connection elements lock together.

In certain embodiments, the distal occluder element is configured to assume a diametrically-reduced configuration when disposed within the tube and a diametrically-expanded configuration when pushed out of the tube. The proximal occluder element may be configured to assume a diametrically-reduced configuration when disposed within the tube and a diametrically-expanded configuration when pushed out of the tube.

The device may further include a locking mechanism configured to hold the distal occluder element to the delivery device after the distal occluder element has been pushed out of the tube. The locking mechanism may comprise a locking sheath with an expandable region and a locking rod extending through the locking sheath. The locking rod may include a locking tip, wherein when the locking tip is pulled back with respect to the locking sheath, it causes the expandable region to expand.

In some embodiments the tube is a needle configured to deliver the occluders percutaneously. In other embodiments, the tube is part of a laparoscopic device, e.g., cannula, and may contain multiple needles or occluder elements. In other embodiments, the tube is present in addition to a needle, and the tube fits within the needle. Each occluder element may comprise a plurality of fingers configured to assume a generally linear shape when in the diametrically-reduced configuration and configured to expand away from each other when in the diametrically-expanded configuration.

In related aspects, the invention provides a method for occluding a hollow structure, or clamping multiple tissue or material layers together. The method involves: (i) providing a multiple-occluder delivery device comprising a hollow tube containing a plurality of occlusion elements; (ii) positioning the tube adjacent to a first position on a hollow structure to be occluded; (iii) advancing the tube through a proximal wall and a distal wall of the hollow structure; (iv) deploying a distal occlusion element from the tube; (v) withdrawing the tube back through the hollow structure; (vi) deploying a proximal occlusion element from the tube; (vii) pushing the distal and proximal occlusion elements together to clamp the hollow structure, thereby occluding the first position; (viii) positioning the tube adjacent to a second position on the hollow structure; and (ix) repeating steps (iii) through (vii) to occlude the second position. In certain embodiments, the method involves inserting the tube percutaneously. In other embodiments the tube can be positioned using laparoscopic imaging or visualization. The imaging modality may be integrated with the occluder delivery tube, or cannula or separate from it.

The method may involve using a multiple-occluder delivery device that includes a hollow tube having a proximal end and a distal end; a plurality of occluders disposed serially within the tube, each occluder comprising a distal occluder element and a proximal occluder element, wherein the distal occluder element is configured to assume a diametrically-reduced configuration when disposed within the tube and a diametrically-expanded configuration when pushed out of the tube and wherein the proximal occluder element is configured to assume a diametrically-reduced configuration when disposed within the tube and a diametrically-expanded configuration when pushed or deployed out of the tube; and a pushing member configured to push an occluder out of the distal end of the tube. The method may further involve using the pushing member to push a distal occluder element out of the tube, such that the distal occluder element expands to its diametrically-expanded configuration; and using the pushing member to push a proximal occluder element out of the hollow tube, such that the proximal occluder element expands to its diametrically-expanded configuration. In other embodiments, the distal occluder may be pushed or pulled out of the tube using the locking rod.

In some embodiments the distal occluder element and the proximal occluder element are separate from but connectable to each other. In other embodiments the distal occluder element and the proximal occluder element are part of a single occluder device. The distal occluder element may comprise a connecting rod comprising a first connection element and the proximal occluder element may comprise a second connection element. When the distal and proximal occluder elements are pushed together, the connection elements lock together.

Some embodiments of the method involve using a device that further comprises a locking mechanism configured to hold the distal occluder element to the delivery device after the distal occluder element has been pushed out of the tube. When the tube is retracted through the distal wall and proximal wall of the hollow structure, the locking member remains coupled to the distal occluder element. The locking mechanism may include a locking sheath comprising an expandable region and a locking rod extending through the locking sheath, the locking rod comprising a locking tip. When the locking tip is pulled back with respect to the locking sheath, it causes the expandable region to expand.

In some embodiments, each occluder element comprises a plurality of fingers configured to assume a generally linear shape when in the diametrically-reduced configuration and configured to expand away from each other when in the diametrically-expanded configuration.

In a related aspect, the invention provides a device for delivering multiple occluders. The device includes a chamber containing a first occluder and a second occluder and a pushing member configured to extend axially through the chamber to deploy the occluders through a distal end of the chamber.

In certain aspects, each occluder comprises a distal occlusion element and a proximal occlusion element. The chamber can be an elongated tube. The occluders can be disposed within the chamber in series. In other embodiments, the device includes a second chamber, substantially similar to the first chamber, situated parallel to the first chamber, and the occluders are therefore disposed in parallel. In some embodiments, the first and second chambers are disposed within a barrel.

### BRIEF DESCRIPTION OF THE DRAWINGS

FIG. 1 shows a prior art varicocele treatment procedure.

FIG. 2 shows a method of varicocele treatment according to the present invention.

FIG. 3 shows a distal end of a delivery device.

FIG. 4 shows a distal end of a delivery device penetrating a hollow structure.

FIG. 5 shows a distal end of a delivery device deploying a distal occluder element.

FIG. 6 shows a locking mechanism.

FIG. 7 shows a delivery device including an occluder pusher.

FIG. 8 shows a distal occluder element deployed on the far side of a hollow structure.

FIG. 9 shows a diagram of the locking mechanism holding a distal occluder element.

FIG. 10 shows the locking mechanism attached to the distal occluder element.

FIG. 11 shows the proximal occlusion element as it moves towards the distal occlusion element.

FIG. 12 shows the proximal and distal occlusion elements moved towards each other to clamp a hollow structure.

FIG. 13 shows occlusion elements and locked together.

FIG. 14 shows occlusion elements and locked together.

FIG. 15 shows the locking mechanism being pulled back into the tube.

FIG. 16 shows the locking rod and sheath being retracted into the tube.

FIG. 17 shows the tube being pulled back from the occluder.

FIG. 18 shows the proximal and distal occluder elements locked together.

FIG. 19 shows a diagram of how the distal and proximal occlusion elements connect together.

FIG. 20 shows another embodiment of how the distal and proximal occlusion elements connect together.

FIG. 21 shows a one-part occlusion device.

FIG. 22 shows the deployment of a single piece occluder.

FIGS. 22A-K show other embodiments of occluders.

FIG. 23 shows a single piece occluder.

FIG. 23B shows a method of manufacturing an occluder.

FIG. 24 shows a multiple-occluder delivery device with multiple locking elements.

FIG. 25 shows a hollow structure occluded by deploying occluders into tissue surrounding the hollow structure.

FIG. 26 shows another embodiment of an occluder delivery device.

FIG. 27 shows another embodiment of a multiple-occluder delivery device

FIGS. 28-37 show a laparoscopic device with six chambers for delivering occluders.

FIGS. 38-41B show views of the delivery device and internal operating and actuation mechanism of the laparoscopic or multi-occluder delivery device.

FIG. 42 shows occluders used for bariatric surgery.

FIG. 43 shows a method of delivering multiple occluders with the present invention.

FIGS. 44-46 show a pushing mechanism for pushing occlusion elements out of a delivery tube.

FIGS. 47-55 show a mechanism for locking proximal and distal occlusion elements together.

FIGS. 56-63 show a mechanism for locking the distal occlusion element to the locking rod.

FIGS. 64A-J show low-profile occluders.

### DETAILED DESCRIPTION

The present invention relates to a delivery device capable of delivery and deployment of multiple occluders for partial or complete occlusion of hollow structures including cavities, ducts, tubular structures, or regions or portions of ducts, cavities and tubular structures. The present invention may for example be used to treat a varicocele by occluding the pampiniform venous plexus. Alternatively, it can be used to occlude a part of the stomach to create a sleeve or pouch that minimally invasively and selectively bypasses sensing regions or guides towards sensing regions in the stomach for weight reduction and weight control purposes. A film can be applied on top surface of the stomach and the occluders deployed through this film, such the film is attached to the outer surface of the stomach, clamped by the proximal occluder fingers, while the distal fingers also clamp the two walls of the stomach together. The film, which may be a polymer, or mesh material, can act to support the occlusion elements and extend the closure forces applied between the occluder elements to the tubular structure, i.e., in this case the stomach. Methods and devices of the invention are also useful for cholecystectomies for closing the main artery and duct and other blood vessels when removing a gallbladder, in laparoscopic, percutaneous, or open surgical procedures. The invention is ideal for any procedure where a hollow structure, cavity, duct, or tubular structure is to be occluded. Devices of the present invention are low cost and so they are ideal as single-use devices for a patient. In some embodiments, however, the device can be reused.

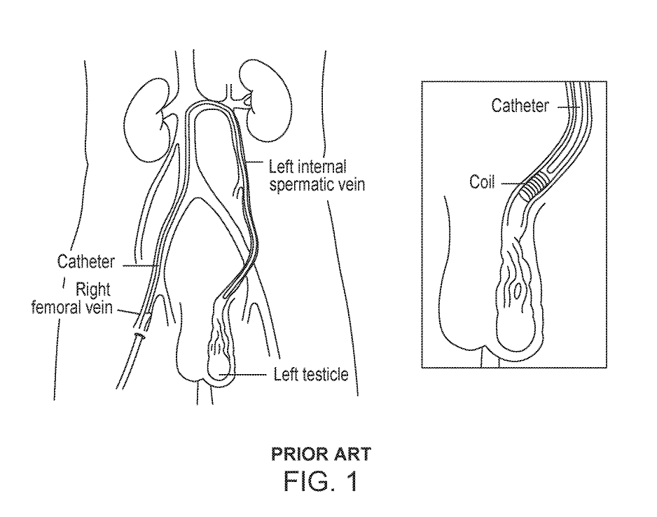

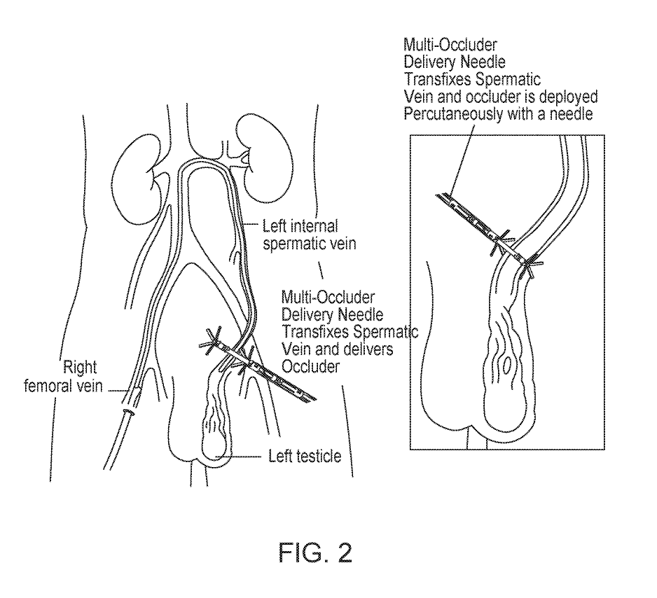

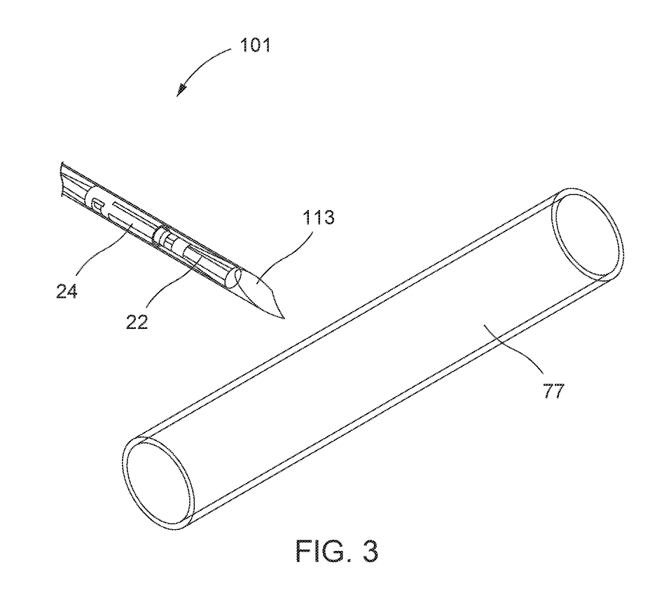

The present invention provides advantages over traditional varicocele treatments. A known varicocele treatment procedure is shown in FIG. 1. A coil is deployed within a vessel by a catheter that has been snaked through the vessel. Unlike that prior art method, the present invention provides occluders that can be delivered percutaneously with a simple syringe-like injection to deploy the occlusion devices to transfix and occlude the vessel, as shown in FIG. 2.

FIG. 2 shows a delivery device of the present invention, which provides a simplified, minimally-invasive treatment of varicoceles by the deployment of an occlusion device to transfix and occlude a vessel. The multiple-occluder delivery device transfixes the spermatic vein and delivers an occluder. As will be explained in greater detail below, the delivery device is configured to percutaneously deliver occluders to other hollow structures in the body as well. A single-occluder delivery device may also be utilized. Laparoscopic delivery of the occluders and visualization may also be envisioned for some embodiments. Any laparoscopic procedures discussed herein may include direct visualization using a camera imager or fiber optics. Visualization may also be enhanced by inflating or pushing away tissue that would otherwise obscure visualization. Inflation may be done with carbon dioxide or another gas. Surrounding tissues can also be inflated to compress the structure to be occluded, thereby easing the occlusion of that structure using the delivery device. Mechanical techniques and means for moving, adjusting, compressing, or positioning the tissue to simplify occlusion of the structure or aperture are also envisioned. The mechanical means may be a clamp with jaws to temporarily clamp or compress the hollow structure prior to occlusion, or pushrods to collapse or reduce the size of the hollow structure.

The present invention combines three key characteristics: the ability of the occluder to clamp, enabling easy and controlled occlusion of hollow structures such as blood vessels; transfixion, which provides anchoring similar to a surgically applied transfixion suture, thereby securing the implant in place and eliminating slippage; and grasping with interweaving between occluding arms, which allows occlusion of both arteries and veins of different size and wall thickness, and ensures an area of hemostasis around the needle entry point.

The occluder and delivery device used in this procedure is of a prototype design that can easily be modified to optimize its performance depending on the circumstance. Although, in an embodiment of the occluder, the combined length from tip-to-tip of two occluder fingers is 5.5 mm, vessels up to 9.5 mm have been occluded with a single occluder. That is due to projection of closure due to the rippling of the tissue produced by the interdigitation of the occluder fingers. In other vessels, placement of two occluder clips successfully occluded these vessels of 1 cm diameter or greater. However, the size and length of the occluder used can be easily modified to match the vessel size, such that single clip vessel occlusion can easily be achieved.

Deployment of the occluders involves minimal patient discomfort and recovery, with only a local injection of anesthetic at the site of the needle injection and or occlusion to minimize patient discomfort. The entire procedural time can be less than 30 seconds per occlusion. This makes such an occlusion method a very cost-effective.

Visualization can also be aided by ultrasound. Delivery tubes of the invention, including needles and laparoscopic cannulas, may be coated with an echogenic or reflective material to enhance ultrasound visualization. Occluder elements can be made echogenic by laser scribing or surface roughening or by coating with echogenic or reflective materials such as polymers.

FIGS. 3 through 17 generally show delivery devices of the present invention in various stages of a delivery procedure.

FIG. 3 shows a distal end of a delivery device 101 positioned near a hollow structure 77 to be occluded. The delivery device includes a hollow tube 113, which may be a needle. The tube 113 contains multiple pre-loaded occlusion elements 22 and 24.

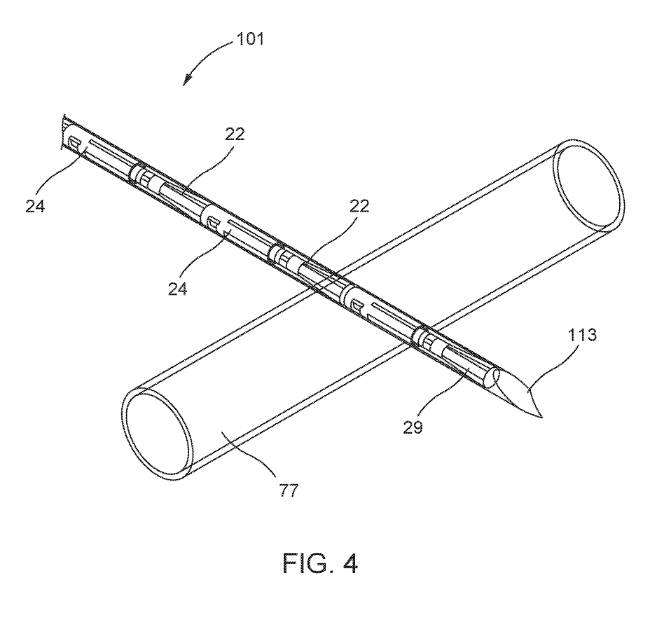

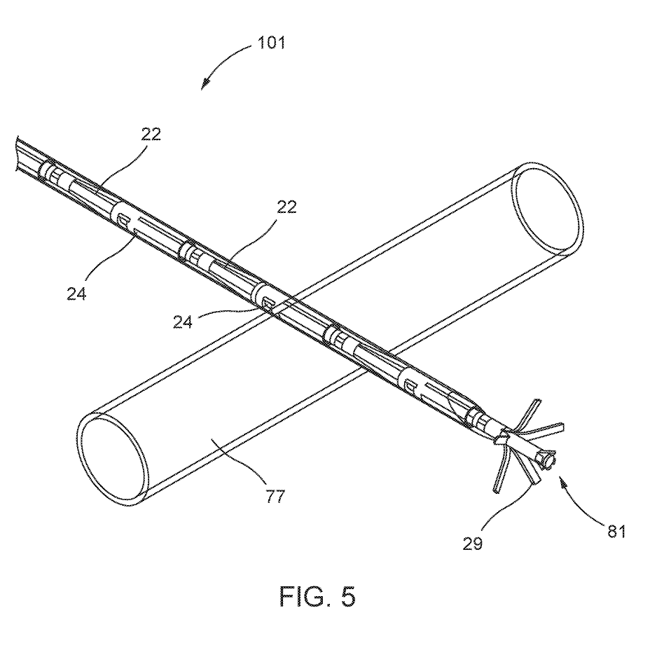

As seen most clearly in FIGS. 4 and 5, the occlusion elements 22 and 24 can be pre-loaded in a serial configuration in the occluder delivery device 101, so that occluders can be delivered sequentially one after the other in close proximity, or at disparate locations of a target structure 77 to be occluded. This multiple occlusion delivery device 101 is configured to deploy multiple occluders without the need to entirely remove the occluder delivery device 101 (i.e., the tube), from a patient's body or laparoscopic port between deployments of subsequent occluders.

As shown in FIG. 4, the hollow tube 113 penetrates and passes through a proximal and distal wall of the hollow structure 77. The tube 113 contains multiple occluder elements 22 and 24. The occluder elements 22 and 24 have expandable fingers 29, which have been collapsed into their diametrically-reduced configuration in order to fit into the tube 113. The proximal 24 and distal occlusion elements 22 are aligned so that their expandable elements 29 (also referred to as fingers or arms) are oriented in the same direction, facing towards the deployment opening of the tube 113 (e.g., needle tip). Other embodiments and configurations where the distal 22 and proximal 24 occluder expandable elements are facing each other in the delivery tube 113 are also possible. Once the delivery tube 113 is positioned at the desired location, the occluder delivery tube 113 is activated to transfix the proximal and distal walls of the hollow structure 77. This movement can be achieved by pushing the tube 113 through the hollow structure 77, or via depressing levers or pressing a button to activate a motor or spring system that moves the tube 113 through the structure 77.

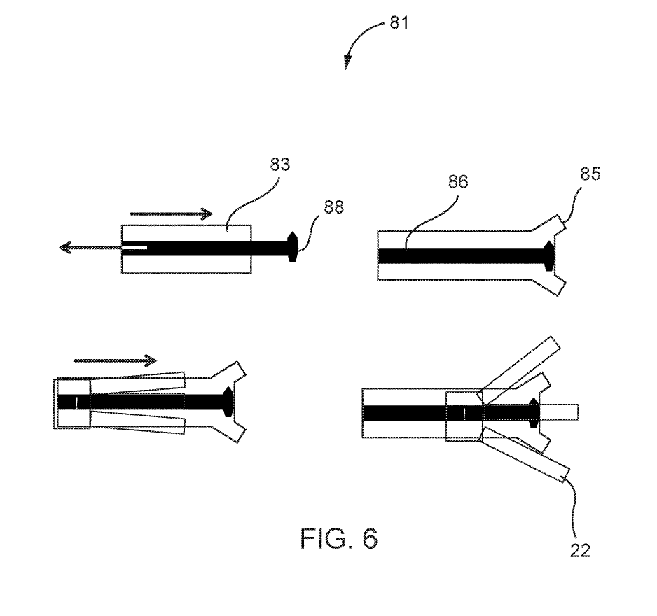

Once the tube 113 is deployed through the distal wall of the hollow structure 77, the distal occlusion element 22 can be pushed out through the distal end of the tube 113. When the distal occlusion element 22 is outside of the tube 113, the fingers 29 expand to the diametrically-expanded configuration, as shown in FIG. 5. The distal occlusion element 22, however, remains attached to the delivery device 101 by a locking mechanism 81, as shown in FIGS. 6-8 and described in greater detail below.

An advantage of the occluder elements configured so that they are aligned in the same orientation with the expandable portion of each of the occluder elements oriented toward the hollow structure to be occluded is that it allows easy removal or retraction of a distal occlusion element if deployed unintentionally in an undesirable location, and to be re-deployed at a desired site, even after full deployment. The delivery device is capable of retracting the distal occluder element back into the delivery tube or by passing the tube over the deployed distal occluder element, thereby re-sheathing the distal occluder element, and compressing the expandable fingers back into the diametrically-reduced configuration.

This can be achieved by pulling up on the locking mechanism, which pulls the distal occluder into the occluder delivery tube. The occluder delivery tube may also be pushed down over the distal occluder element to collapse the distal occluder and re-sheath.

Returning to FIG. 5, the distal occlusion device 22 is deployed from the tube 113 and held in place by the locking mechanism 81. Details of the locking mechanism 81 are shown in FIG. 6. The locking mechanism 81 is configured to fit through a coaxial lumen in the distal occlusion element 22. The locking mechanism 81 includes a locking sheath 83 and a locking rod 86. The locking rod 86 can be a guidewire. At the distal end of the locking rod 86 is the locking tip 88, which is an enlarged region of the locking rod 86 or guidewire. When the locking rod 86 is moved proximally with respect to the locking sheath 83 so that the locking tip 88 contacts the distal end of the locking sheath 83, it causes the distal end of the locking sheath (i.e., the sheath expansion region 85) to expand.

The expansion of the sheath expansion region 85 prevents the distal occluder 22 from falling off the locking mechanism 81. The expansion of the locking sheath can also be done when the sheath expansion region 85 is inside the distal occluder 22, thus fixing the position of the distal occluder 22 on the locking mechanism 81.

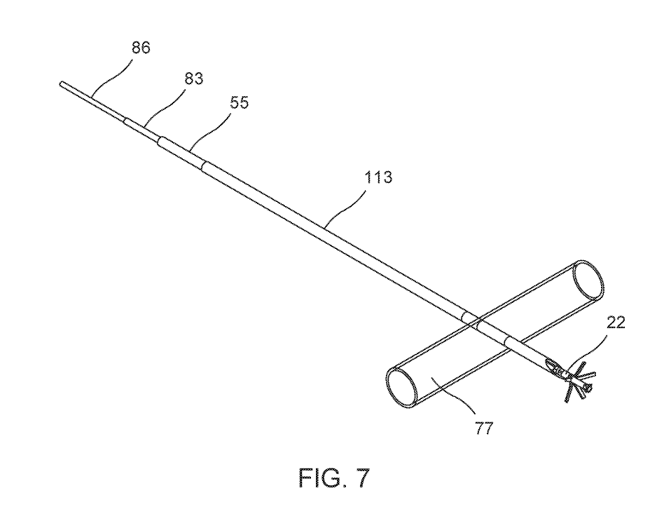

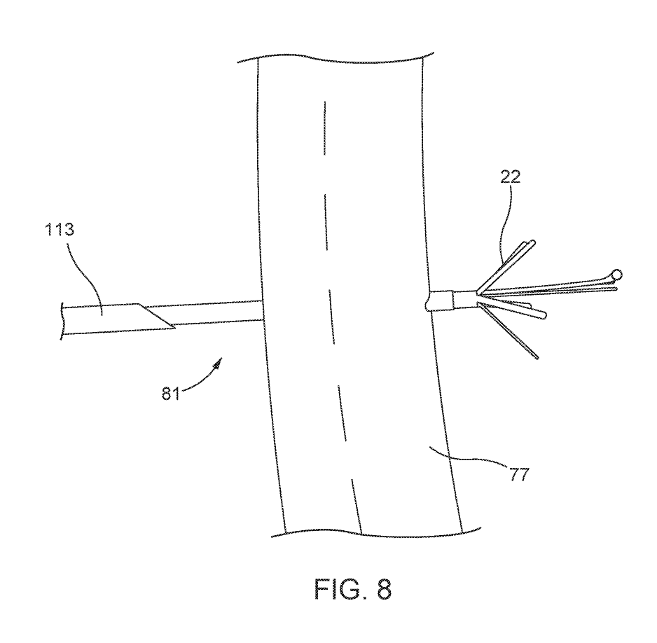

In an embodiment of the invention, an occluder pusher 55 is used to push the distal occluder 22 out of the needle, as shown in FIG. 7. In this embodiment, the occluder pusher 55 has a coaxial cylindrical configuration, and when activated pushes the stacked occlusion elements (not shown) which pushes the distal occlusion element 22 out of the tube 113.

The locking mechanism 81 is activated by moving the locking sheath 83 forward away from the delivery device handle (not shown) and moving the locking rod 86 backwards towards the delivery device handle. By moving the sheath 83 and rod 86 in opposite directions relative to each other, locking or release of the distal occlusion element 22 can be achieved. The locking sheath expansion region 85 expands, thereby acting as a lock to prevent the distal occluder 22 from moving beyond the end of the locking rod 86. In another embodiment, the distal occlusion element 22 can be locked onto the locking mechanism 81 by the force the expanded locking expansion region 85 exerts on the distal occluder element 22. In another embodiment, the distal occluder 22 locks onto the locking rod 86, and is released from the locking rod 86 when a proximal occluder 24 locks with the distal occluder 22, thereby unlocking the distal occluder 22 from the locking rod 86 by pushing on the locking element region between the distal occluder 22 and locking rod 86.

FIG. 8 shows a distal occluder element 22 deployed on the far side of a hollow structure 77. The distal occluder element 22 is held to the delivery device 101 by the locking mechanism 81. The delivery tube 113 has been pulled back through the hollow structure.

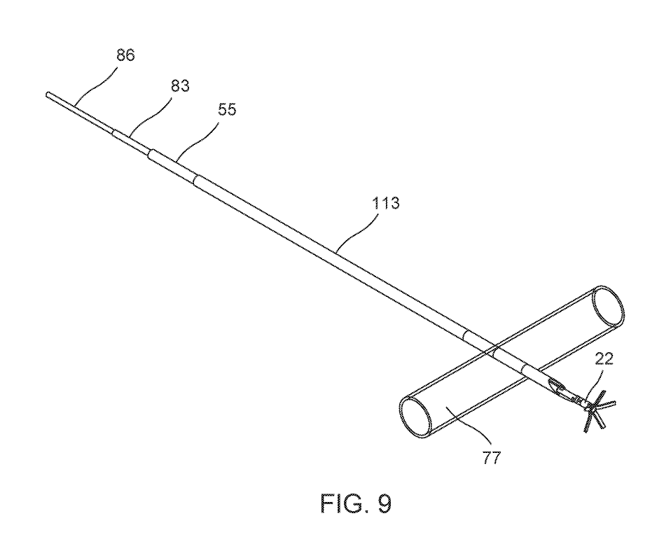

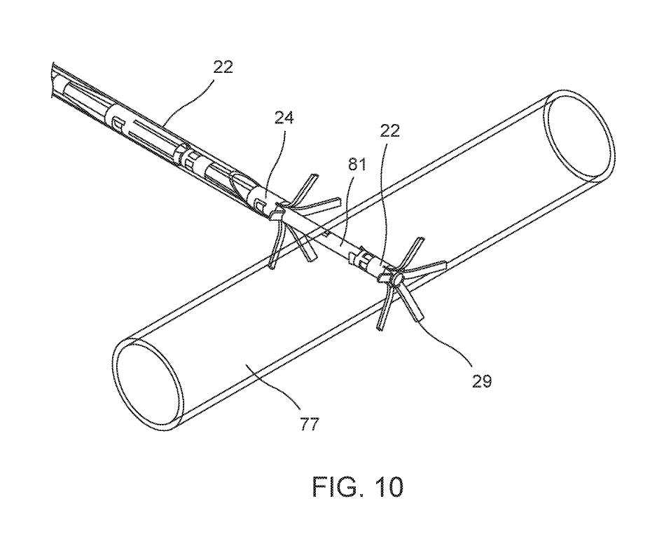

FIG. 9 shows a diagram of the locking mechanism 81 (including locking sheath 83 and locking rod 86) holding the distal occluder element 22, which has been deployed and expanded. FIG. 10 shows the locking mechanism 81 remains coupled to the distal occlusion element 22 on the far side of the hollow structure 77, while the tube is moved back until it is outside the structure to be occluded 77, whereupon the occluder pusher (not shown) is pushed forward to deploy the proximal occluder element 24 on the near side of the hollow structure 77.

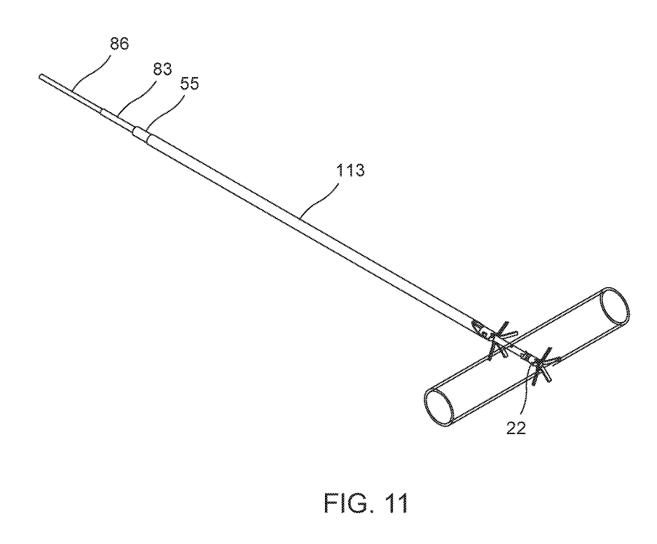

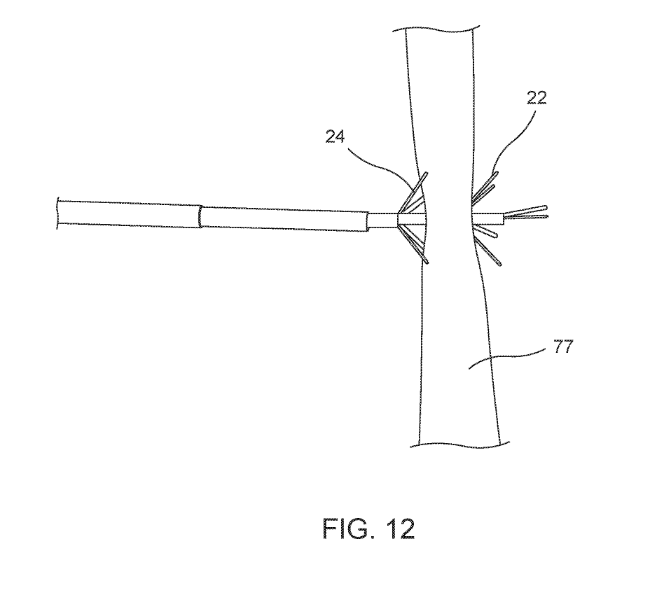

FIGS. 11-14 show various views of the distal and proximal occlusion elements as they move towards each other and lock together. The locking element may be moved up away from the needle tip pulling the distal occlusion element towards the proximal element, and the occluder pusher is moved down in the direction of the needle, pushing the proximal occluder element towards the distal one. Once the distal and proximal occlusion elements are in contact they lock by means of connection elements on each occluder. Once locked, the locking mechanism is released by pushing the inner locking rod forward in the direction of the needle tip

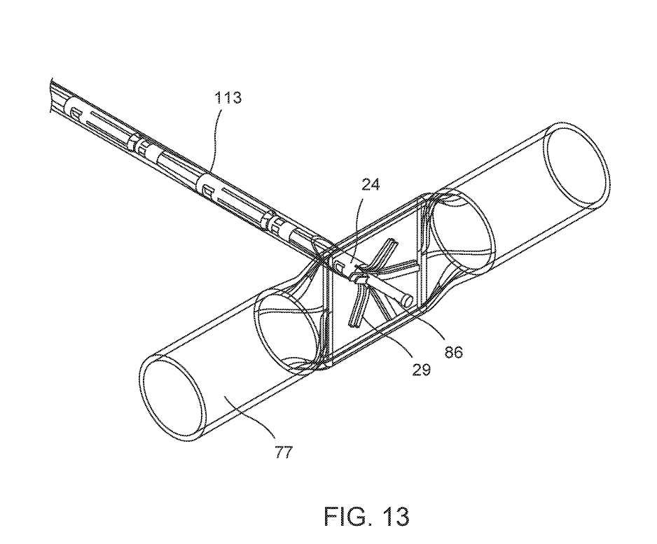

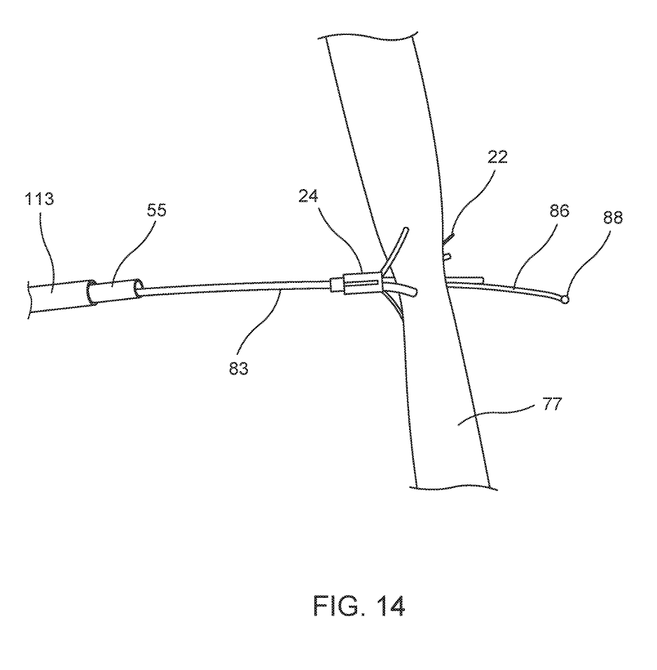

FIG. 11 shows the proximal occlusion element 24 moved towards the distal occlusion element 22, where they will lock the distal 22 and proximal 24 elements together to clamp the hollow structure 77. The movement of the distal 22 and proximal 24 elements is actuated by pulling the locking mechanism 81 back and pushing the occluder pusher 55 forward. FIG. 12 shows another view of the proximal 24 and distal 22 occlusion elements moved towards each other to clamp the hollow structure 77 and lock together. FIG. 13 shows a diagram of the occlusion elements 22 and 24 locked together. Once they are locked together, the locking mechanism can be released by moving the locking rod 86 forward, causing the expansion region to reduce in size. The sheath can then be drawn back through the occlusion elements, followed by the locking rod 86. FIG. 14 shows another view of the locking rod 86 moving forward once the occlusion elements 22 and 24 are locked together.

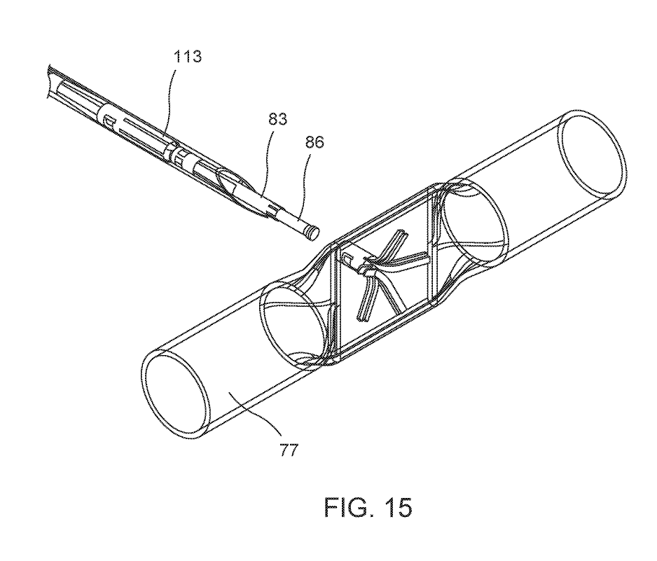

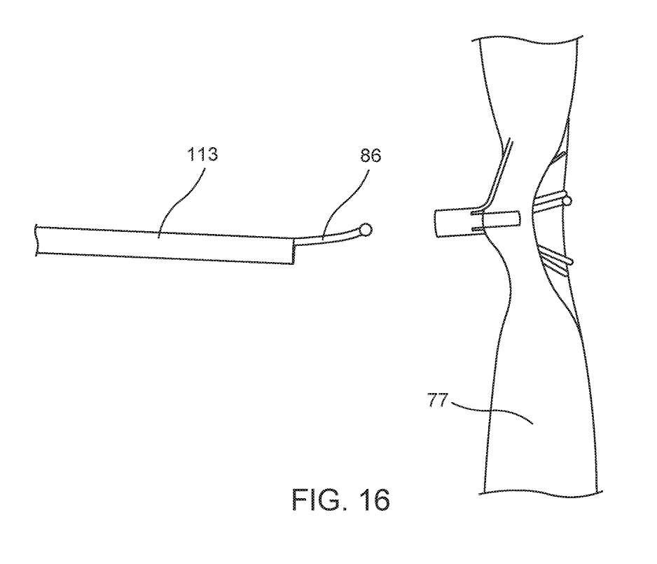

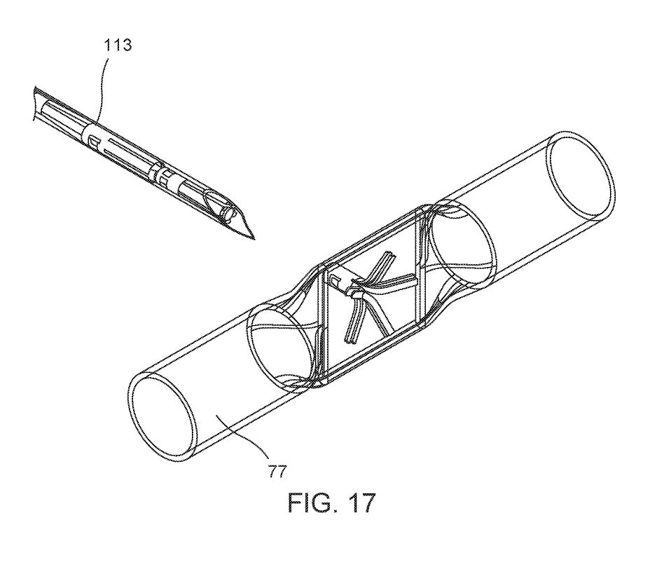

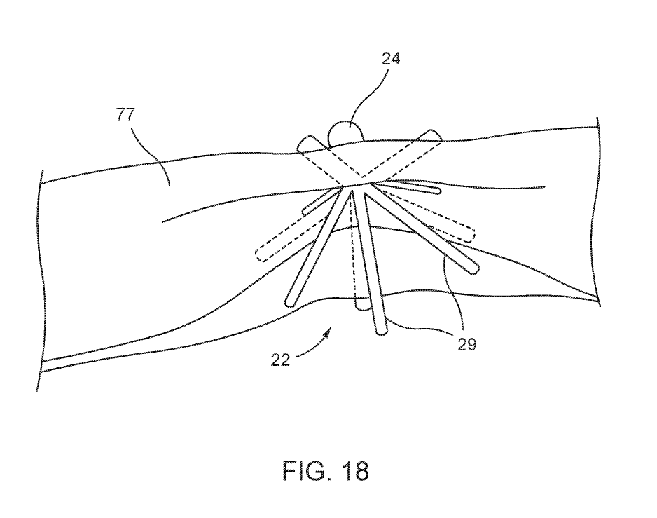

FIGS. 15-17 show the locking mechanism 81 being pulled back into the tube 113, leaving the occlusion elements 22 and 24 locked in place transfixing the hollow structure 77. FIG. 15 shows the locking rod 86 and sheath 83 after they have been retracted from the deployed occluder. FIG. 16 shows another view of the locking rod 86 and sheath 83 being retracted into the tube 113. FIG. 17 shows the tube 113 being pulled back from the occluder. FIG. 18 is a view of the proximal and distal occluder elements locked together in place transfixing a hollow structure 77.

Other possible embodiments and constructions are also envisioned. Another embodiment of the invention does not require the locking mechanism to have a sheath. In one embodiment, the locking mechanism simply consists of a rod with a fixed enlarged region, e.g., bulbous, or sphere, at the distal end. In an embodiment of the invention, the force that the occluder pusher exerts on the occluder elements, allows the occluder elements to be pushed and expand over the bulbous region and be deployed.

In another embodiment of the invention, the locking rod contains an expandable bulb. There are various ways the tip of locking rod can be made to expand. It may be an inflatable and deflatable structure (e.g., a balloon) with defined dimensions. The inflation may be done by gas (e.g., nitrogen or air) or liquid (e.g., saline). In another embodiment it may be an expandable construction of various designs consisting of expandable elements whose release is controlled at the proximal end of the rod by the operator, for example a bulb with tines that expands by rotation, or bumps, or the like. The bulb can also be inflated to multiple sizes, and in one embodiment, it is inflated initially significantly, so that it acts to separate the hollow structure from tissue beneath the vessel, stretching and making space for the deployment of the distal occlusion device so that it does not penetrate or impact the tissue beneath. The bulb can then be deflated to a smaller size to act as a stop.

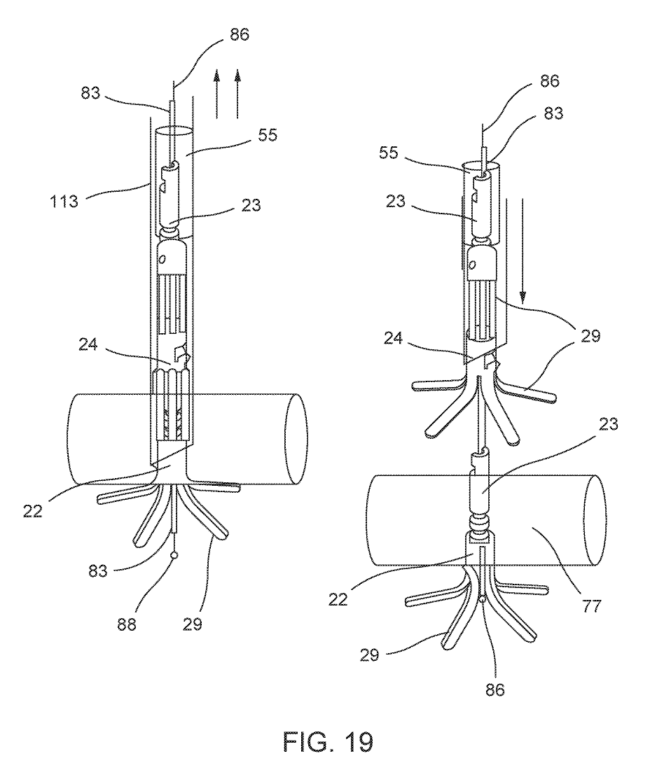

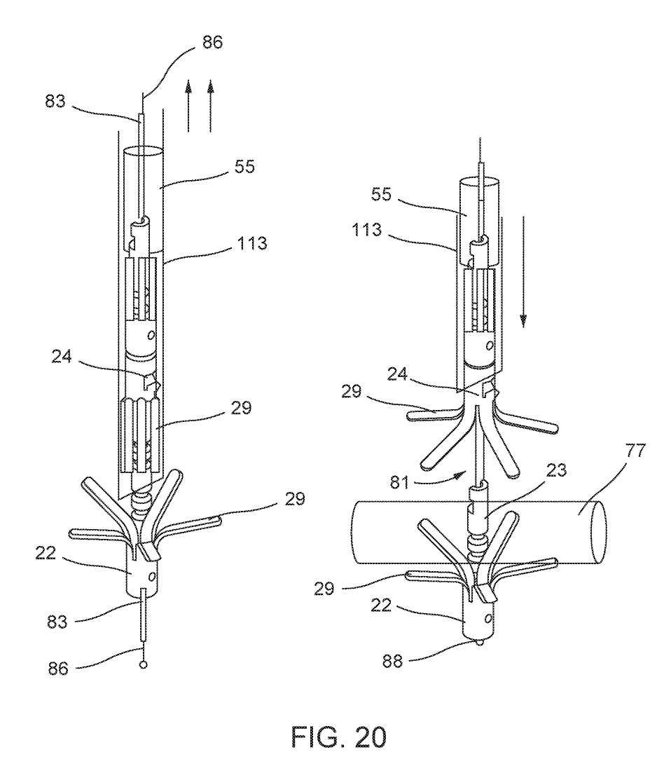

FIGS. 19 and 20 show details of how the distal 22 and proximal 24 occlusion elements connect together. The distal occlusion element 22 includes a connecting rod 23. The connecting rod 23 is hollow and allows the locking mechanism 81 to pass through in a similar manner to other embodiments and illustrations of the inventions described throughout this application. The connecting rod 23 may be made of the same material or a different material to the distal 22 or proximal 24 occlusion elements. The connecting rod 23 is configured to be attachable to the proximal occlusion element 24.

In the configuration of FIG. 19, the distal 22 and proximal 24 occlusion elements are oriented so that their fingers 29 open in the same direction. In the embodiment of FIG. 20, the proximal and distal occlusion elements are oriented to open opposite to each other. In other embodiments the occluders consist of tines or structures other than fingers, capable of applying force and pressure to the tissue to at least partially occlude the hollow structure.

As shown in FIG. 19, multiple occlusion elements 22 and 24 are loaded into a delivery tube 113 or needle. In some embodiments, an occluder holding tube is located within a needle. The fingers 29 of each occlusion element touch the occlusion element in front of it. The distal occlusion element can be pushed out of the delivery device 101 using the occluder pusher 55, whereupon the fingers 29 or tines are opened. The locking rod 86 and locking sheath 83 can be pulled toward the distal occlusion element 22, and then activated by pushing the locking sheath 83 to expand inside of the distal occlusion element 22, thereby locking it to the locking rod 86. The delivery tube 113 may be pulled back or the occluder pusher 55 may be moved forward to deploy the proximal occlusion element 24. Then the occlusion elements 22 and 24 can be pushed together to connect together via locking elements located on each. When the two occlusion elements are locked together transfixing the hollow structure 77, they clamp the hollow structure together and occlude it.

In the embodiments of the current invention where the fingers 29 are aligned in the same orientation for both the proximal 24 and distal 22 occluder, such as the embodiment shown in FIG. 19, the occluder fingers 29 are equal to or longer than the connecting rod 23 of the distal occlusion device, to ensure that the distal and proximal occlusion elements do not lock inside of the delivery tube 113.

FIG. 20 shows an embodiment similar to that shown in FIG. 19, except the fingers 29 of the proximal 24 and distal 22 occlusion devices are facing each other. In this embodiment, the length of connecting rod 23 portion of the distal occluder 22 can be the maximum of the length of the distal and proximal fingers combined.

It should be noted that in some embodiments, the distal occlusion element is purposely embedded in tissue surrounding the structure to be occluded, so that the fingers of the distal occlusion element are not directly touching the structure to be occluded, avoiding any possible damage from the distal occlusion element, while the surrounding tissue compresses the structure to be occluded. Similarly, the proximal occlusion element can be deployed in tissue surrounding the structure to be occluded, so that its fingers are not in direct contact with the structure to be occluded. The two occlusion elements are then locked together and the structure is occluded. This concept can be applied to all embodiments disclosed herein.

Thus far, the occluder has been defined as a two-part device having separate proximal and distal occlusion elements configured to connect together to transfix a hollow structure. However, the invention also provides a one-part occluder that can be deployed in much the same way as the two-part device. Like the two-part occluder, a one-part occluder still has a proximal and distal end (which can be described as a proximal occlusion element and a distal occlusion element). A one-part occluder can be made of various materials, such as nitinol, shape memory material, super elastic material, or the like. It may be made of stainless steel. It can be made of superelastic or plastic material. It may be primarily metallic, or polymeric, or ceramic.

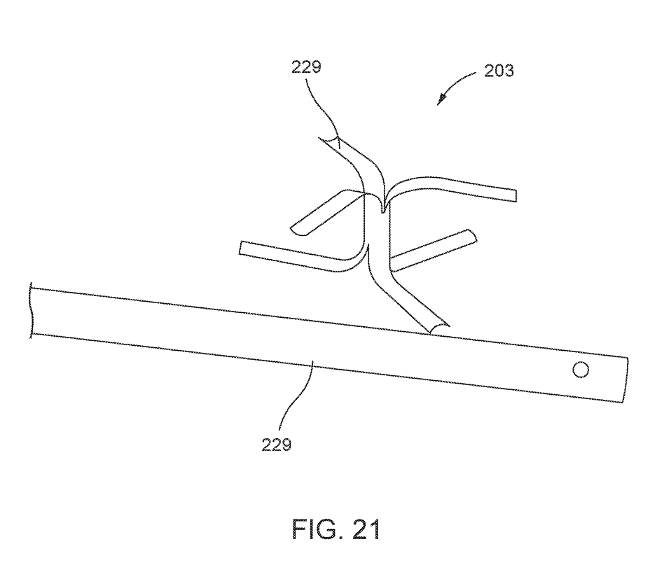

FIG. 21 shows a one-part occlusion device 203. The one-part occluder 203 has a connection part that may be a partial cylindrical structure, with slits in a portion of the occluder to form fingers 229 or tines that are set to deform into a diametrically-expanded configuration when released from the delivery tube 213. It may have an open region in its center to accommodate a guidewire or locking mechanism, expansion sheath, and the like. In some embodiments, the fingers 229 expand in response to a change in temperature. The fingers 229 provide clamping in the generally the same way as the fingers of two-part occluders described above.

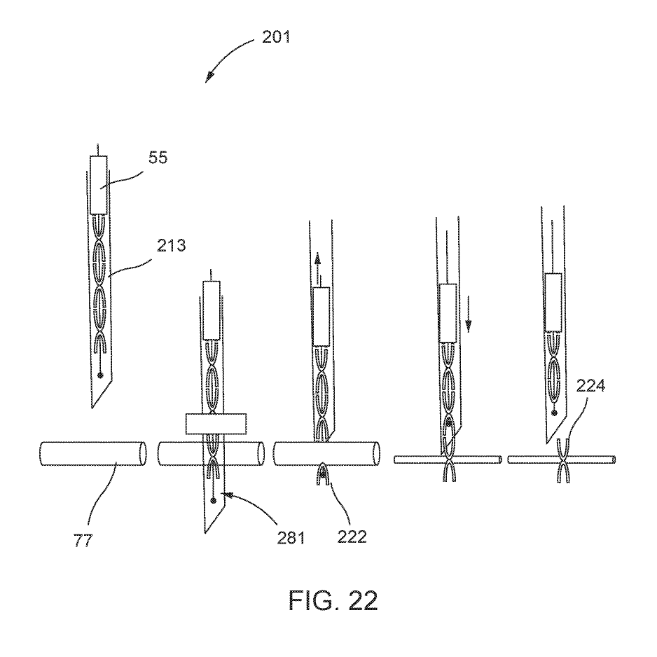

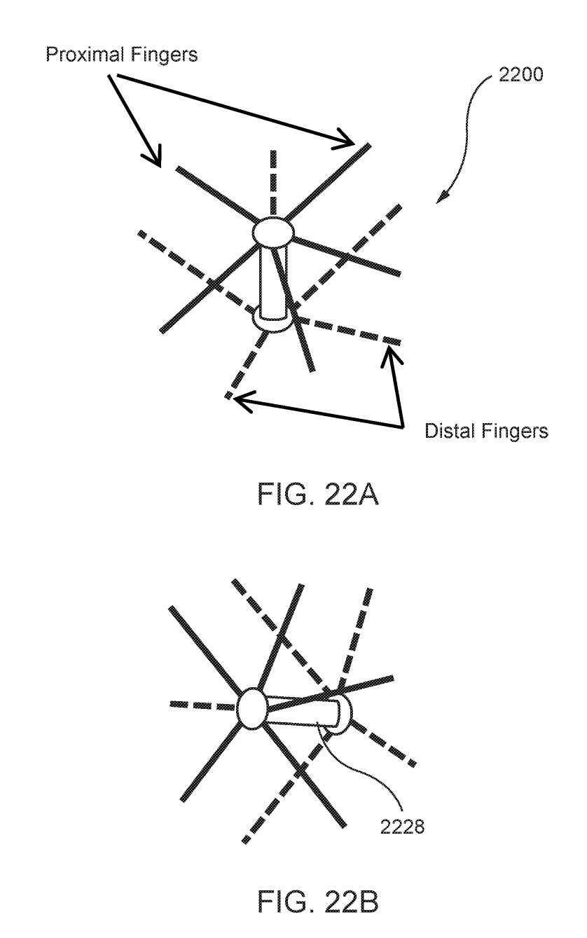

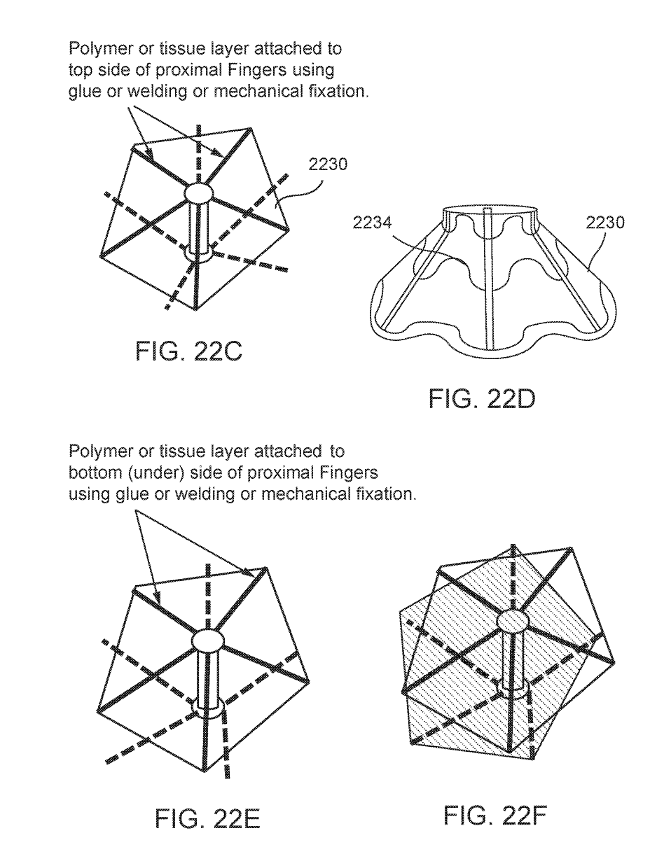

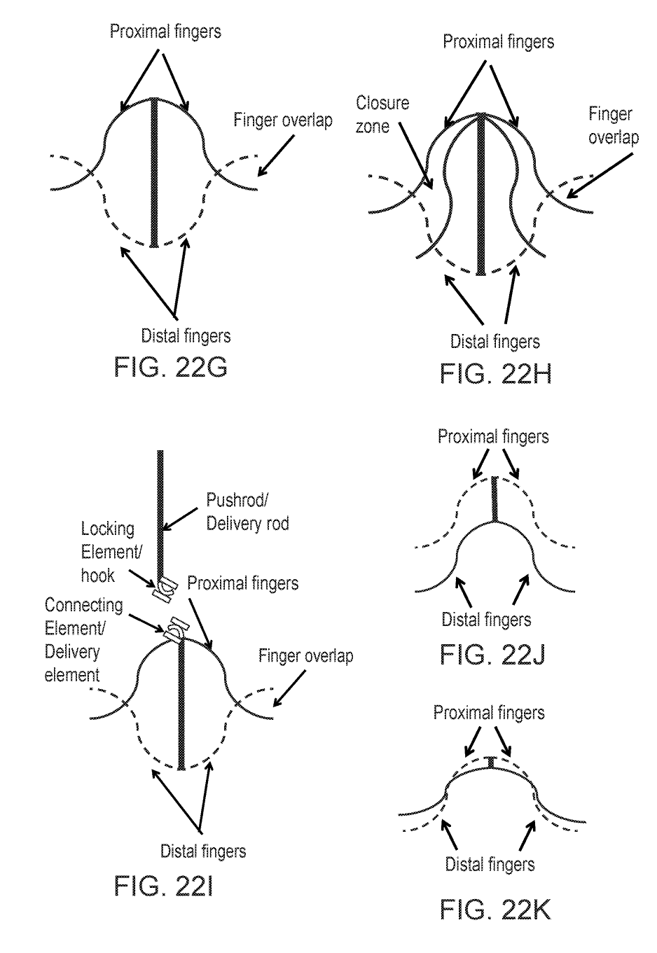

FIG. 22 shows the deployment of a single piece occluder 203 using a multiple-occluder delivery device 201. The tube is passed through the hollow structure 77. The distal end 222 of an occluder is pushed out while the tube 213 is retracted back through the hollow structure 77. The locking mechanism 281, including a locking rod or guidewire, holds the occluder 203 in place. The pusher then pushes the proximal end 224 of the one-part occluder 203 out through the tube 213, leaving the occluder 203 in place transfixing the hollow structure 77. The multiple-occluder delivery device 201 can include a tissue protector element to protect tissue lying beneath the hollow structure to be occluded. The fingers of the occluder element, typically will expand radially beyond the amount they were when in the delivery tube, as shown in the fully expanded state of FIG. 22. A. Note, occluders of FIG. 22 may have only two proximal fingers or may have more, e.g., 5, as shown in FIG. 22A.

A two part occluder has an advantage that positioning of the two parts is highly precise, and the clamping is accurate, whereas a single part occluder provides for less control of deployment of proximal and distal fingers.

FIGS. 22A and 22B show two different views of a single-piece occluder 2200 with a plurality of fingers. The occluder is in its deployed configuration. Typically, the occluder will have approximately five fingers, where the proximal fingers are designed to interdigitate with the distal fingers, and vice versa. Interdigitation causes any material or tissue clamped between them to assume a primarily “wavy” configuration, project closure forces beyond the physical ends of the occluder fingers, and to provide a roughly circular compression zone surrounding the puncture hole where the occluder was delivered for transfixion. In an embodiment of the invention, when the fingers are deployed or released, the tips of the distal fingers project above the plane formed by the proximal fingers, and the tips of the distal fingers, project below the plane formed by the distal fingers. The fingers may be straight or curved in a non-linear manner with their spring constant tailored for the tissue or tubular structure or material to be clamped or occluded. In this embodiment the singe occluder is flush with the fingers (although the fingers bend down in a linear manner or a non-leaner manner) to exert force on the tissue or aperture sandwiched there between. The low profile connection element 2228 between the proximal and distal fingers allows for deployment of the occluder close to the surface of the skin. In other embodiments the single occluder may have a hook element to attach it to a delivery rod that may hold it until deployed.

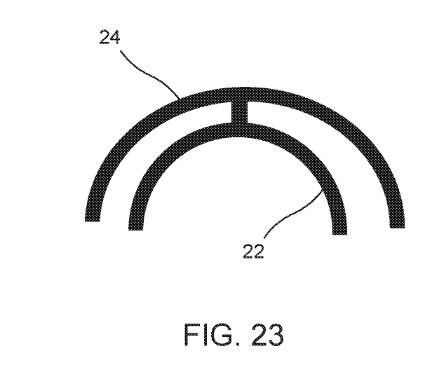

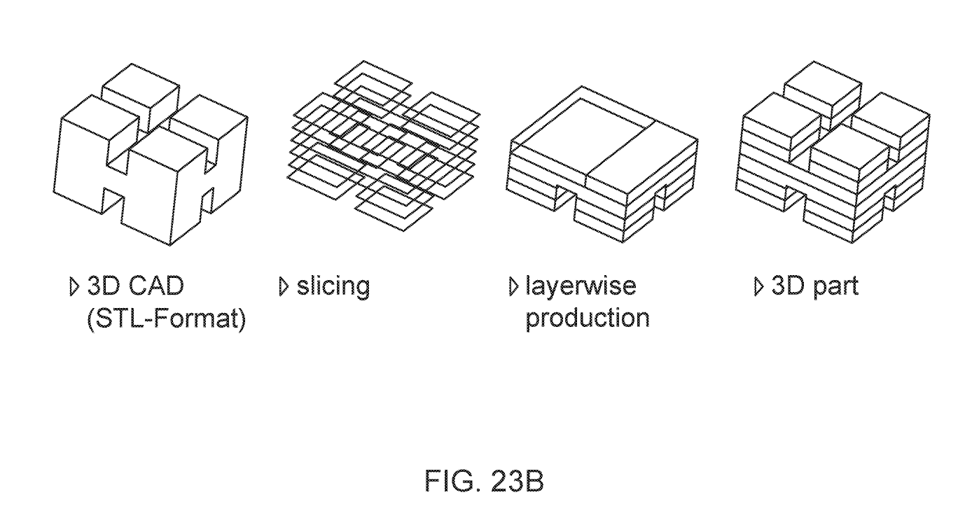

In some embodiments, the single piece occluder can be made out of two pieces that are welded, soldered, glued, or epoxied together at their intersection to form a one-part occluder. The proximal 24 and distal 22 portions of the single occluder can expand to be C-shaped and curve onto themselves, as shown in FIG. 23. In other embodiments, the single piece occluder can be formed as a single element, either starting with a single material and carving, cutting, etching or shaping it appropriately, or by additive manufacturing, 3-D printing, or depositing successive layers of material to build up the shape and structure of the desired device. The deposition or formation may be done at a first condition, for example, at a reduced temperature condition, whereupon heating the material causes the device formed to take on a different shape, structure, or the like.

Occlusion elements may be made using Stereo-lithography, additive printing methods where the fingers and post are deposited in their relaxed state, and then cooled so that the fingers collapse so they can be loaded into the delivery needle. They can also be made by injection molding, and may consist of polymers, metals, composites and the like. FIG. 23B shows a typical additive deposition process, where successive layers of material are deposited. They may be metals such as nitinol or bio-degradible magnesium or polymers. In this way, the single part occluder or two part occluder can be manufactured. They can either permanently occluder a tubular structure, or after they have been present on the tubular structure they can degrade, but the occluded structure can still remain closed due to fibrosis. The fingers can be deposited from nitinol while the connector element from another material, or all of the occluder can be made from nitinol.

In another embodiment of the invention, either the single piece occluder or the two piece occluder may have supporting tissue or polymer or material, pre-attached to the fingers to form a webbing, to assist in further distributing the closing force, constraining the tissue of the hollow structure to be occluded, delivering particular chemicals or drugs in a time-release manner, or to hold the tissue down, and to provide better sealing to prevent blood bile or lymph material leakage.

The webbing material may be coated on top of the fingers, or below the fingers as shown in FIGS. 22C-F.

FIG. 22C shows a webbing 2230, which can be a polymer or tissue layer, attached to the top side of the proximal fingers. The layer 2230 can be adhered using glue, welding, or mechanical fixation. FIG. 22D shows an embodiment where the polymer coating 2230 is ePTFE or silicone. The device may additionally have ribs 2234 extending between the fingers. FIG. 22E shows a view from the proximal side of an embodiment where the fingers of the proximal side are coated with a webbing material. The polymer or tissue layer may also be attached to the distal fingers of a one- or two-part occluder. FIG. 22F shows a polymer or tissue webbing on both the proximal and distal sets of fingers to contain any tissue or material from the structure clamped between, or distribute the force of the clamping fingers more uniformly. The webbing may be hermetic or selectively permeable.

Similar embodiments are also envisioned for the two-part occluder. The two-part occluder proximal and or distal fingers may be coated with a webbing material similar to the single occluder element. The webbing can be collapsed along with the fingers similar to an umbrella folding, to fit within the delivery needle.

The webbed occluder devices are also usefule for sandwiching tissue or closing an opening such as a Patent Foramen Ovale (PFO).

FIGS. 22G-K show cross-section views of occluder elements with overlapping or interdigitating fingers. FIG. 22G shows a single occluder element where three or more pairs of proximal fingers and distal fingers curve away from the attachment region, which may be rigid or elastic. The fingers overlap, but do not necessarily contact each other. FIG. 22H shows the fingers arranged in an opposing configuration. The multiple fingers of the single occluder element interdigitate and provide a closure zone or region around the deployment hole to seal the hole and prevent blood, bile or other materials from flowing out of the transfixion hole. FIG. 22I shows an attachment element that may be part of single occluder element or attached to single occluder element to handle, position, or deploy the single occluder element. FIG. 22J shows a cross section of another embodiment of the single piece occluder, where the fingers, three or more, are curved and the fingers are arranged in an aligned configuration. The central post may simply be a connection point, or may be an elastic or rigid post. FIG. 22K shows a cross section of another embodiment of the single piece occluder, where the fingers are non-linear, and the proximal and distal finger planes overlap.

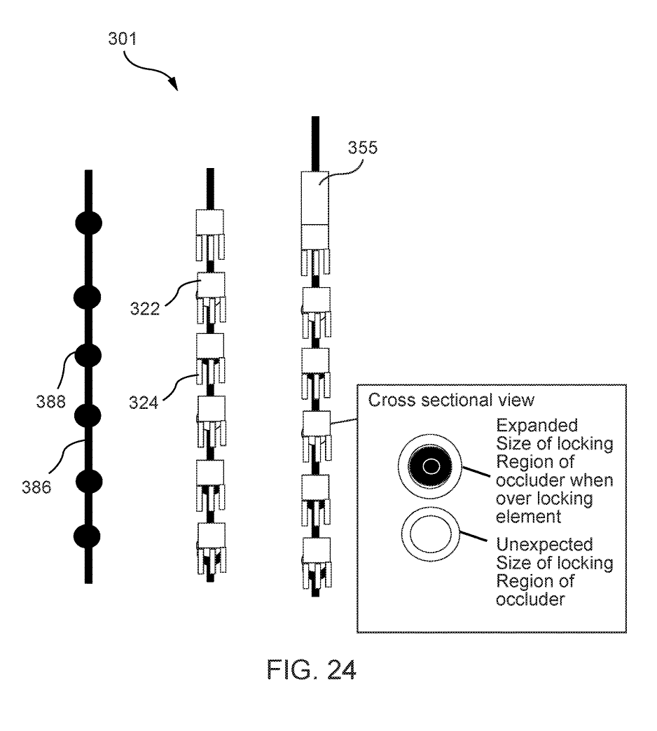

Turning now to FIG. 24, another embodiment of a multiple-occluder delivery device is shown. Multiple-occluder delivery device 301 includes a locking rod 386 that contains multiple locking elements 388 (similar to the locking tip 88 described above, except not located at the distal end of the locking rod). The various occlusion elements 324 and 322 can be mounted on the locking elements 388. The occluder pusher 355, applies force to move one of the occluder elements to the next locking element position, and forces the very last occlusion element out of the delivery tube (not shown), to open up to its diametrically-expanded configuration and be deployed. In an embodiment of the invention, the proximal 324 and distal 322 occlusion elements are made up of sufficiently thin cylinders of nitinol or other materials that they can expand when pushed with sufficient force over the locking elements 388, sufficiently to be pushed past the locking mechanism. However, without applying the force of the pusher 355, the occlusion elements do not move on their own. The occluder elements 322 and 324 and locking rod with elements 388 and 286 will typically be housed within a needle or tubular housing constraining them mostly in a compressed state, with the fingers generally in the closed state. In another embodiment of the invention, locking elements 388 may be made of a soft metal or polymer, allowing them to be compressed, and the occlusion elements can be hard and incompressible.

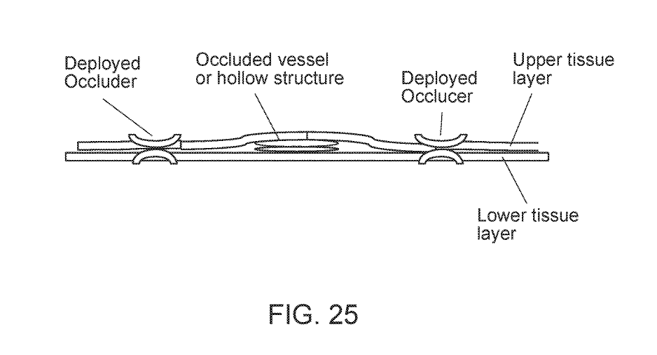

Using any of the multiple-occluder delivery devices described herein, a hollow structure can be occluded by deploying occluders into tissue surrounding the hollow structure, without penetrating the hollow structure itself. FIG. 25 shows two occluders deployed near a vessel to be occluded. The occluders can be any of those described herein, and can be delivered by any of the delivery devices and methods described herein. As show in FIG. 25, the occluders do not penetrate the vessel. They exert a force to the surrounding tissue to close the vessel. The occluders clamp and pull the tissue together and act to occlude the hollow structure located between the tissue layers. In another embodiment, the occluders can be sufficiently close, but not ligating the hollow structure, so that the physical compression of the tines or fingers acts to physically and locally compress the hollow structure closed. The embodiment shown in FIG. 25 may be especially useful in treatment of varicoceles, where it is not necessary to occlude the vessel, but rather constrict the blood flow. By deploying two occlusion devices on either side of the varicocele, reduced flow can be achieved, without damaging or injuring the vein.

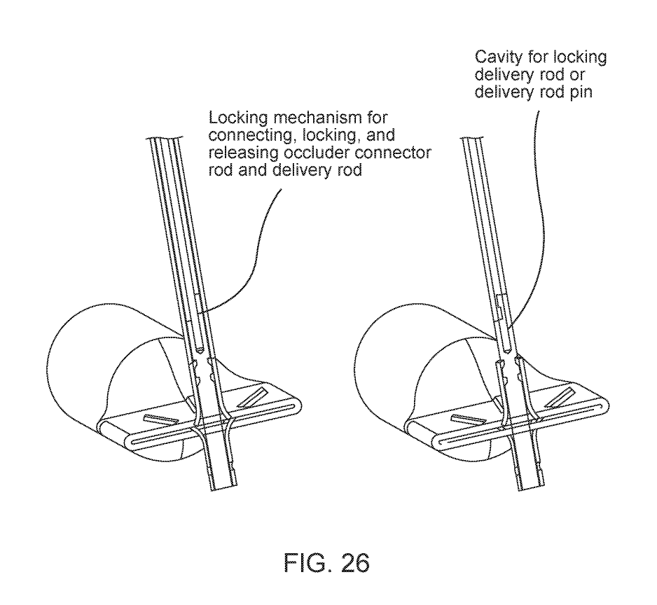

FIG. 26 shows another embodiment, wherein a delivery rod and the connecting rod are reversibly attached, by inserting a delivery rod pin, or simply the delivery rod, into a clasp region between the delivery rod and connecting rod.

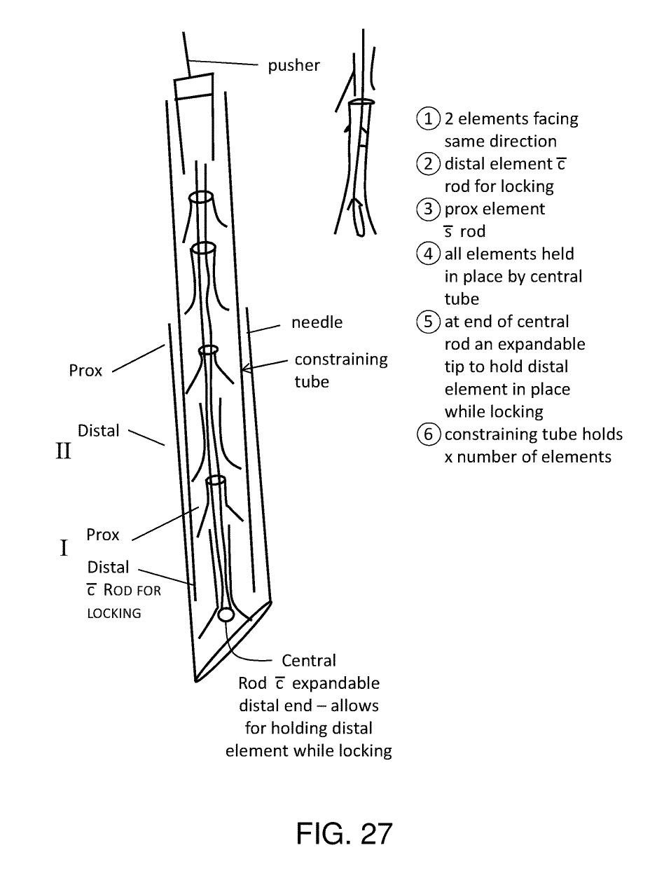

FIG. 27 shows another embodiment of a multiple-occluder delivery device. Distal and proximal occlusion elements are lined up in the delivery tube facing the same direction. As described above, the distal occlusion element includes a connecting rod, and the proximal occlusion element does not include a connecting rod, but is configured to connect to the connecting rod of the distal element. In this embodiment, all of the occlusion elements are held in place by a central rod running through them. The distal end of the central rod has an expandable tip for holding the distal-most occlusion element in place while locking the occlusion elements together during deployment.

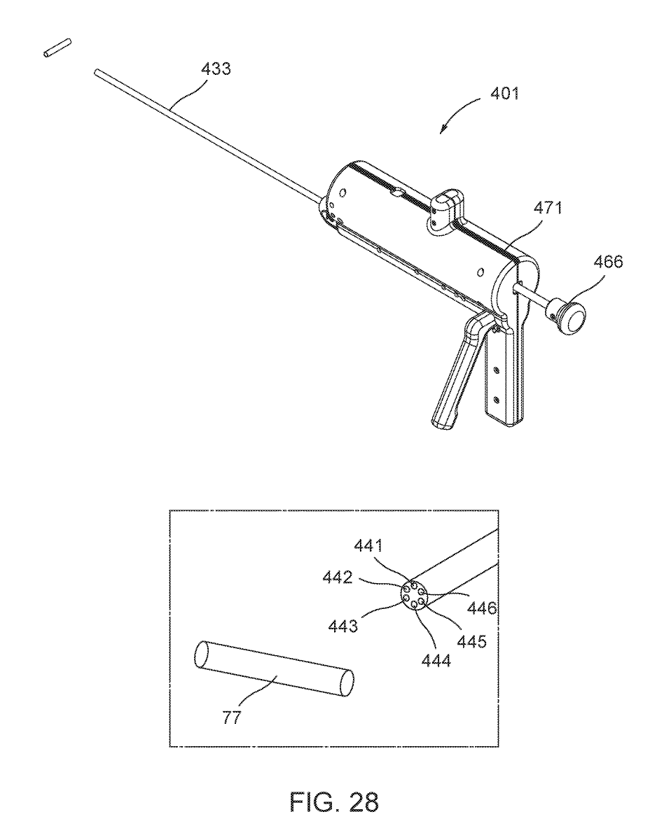

FIGS. 28-37 show another embodiment of a multiple-occluder delivery device 401 where the multiple occlusion elements are held in parallel, rather than in series, in a six-chamber barrel 433. In the embodiment shown, six occluders may each be contained within their own delivery tube or needle. In certain embodiments, the diameter of the delivery tube can have a diameter less than or equal to 10 mm, or less than or equal to 5 mm, to fit into a standard laparoscopic port.

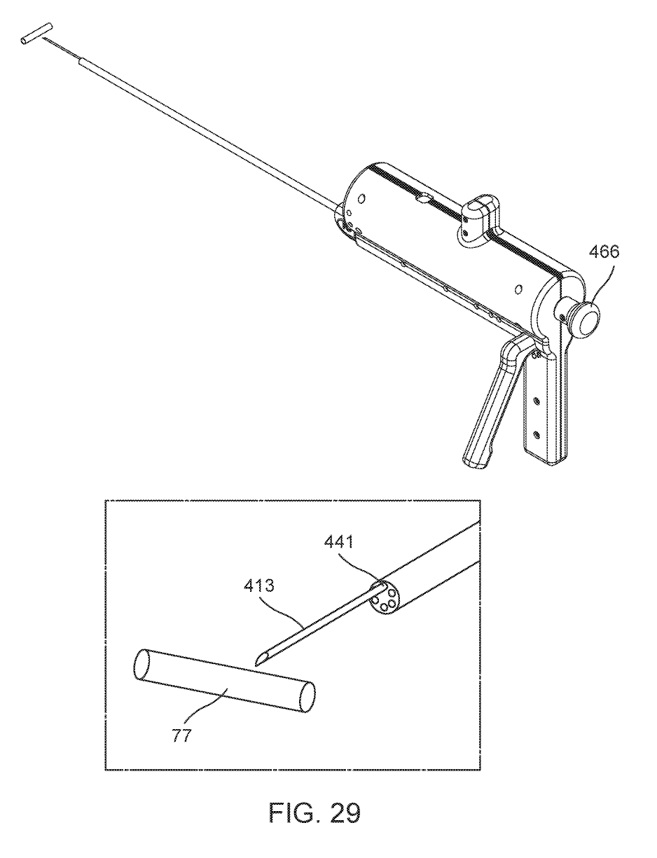

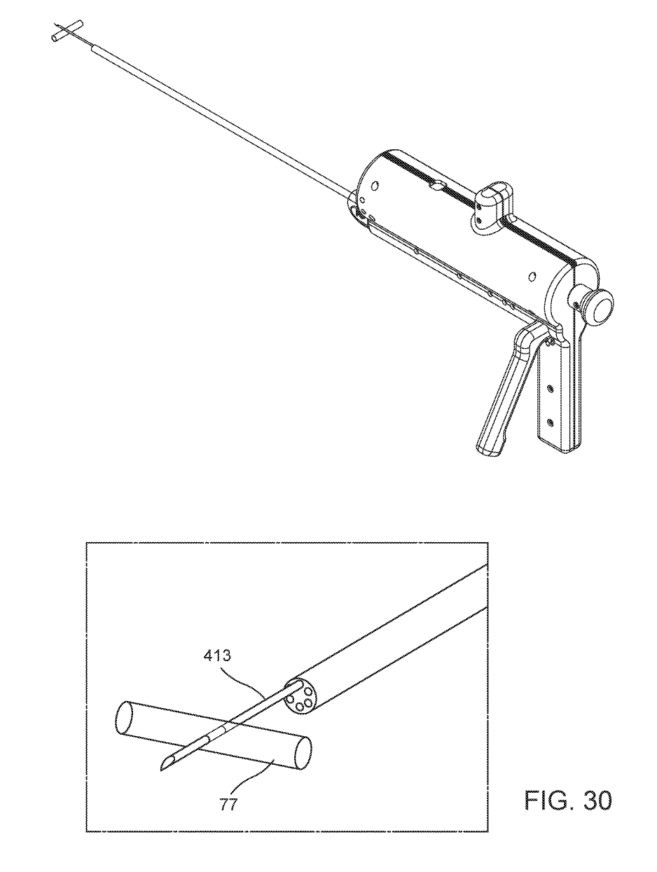

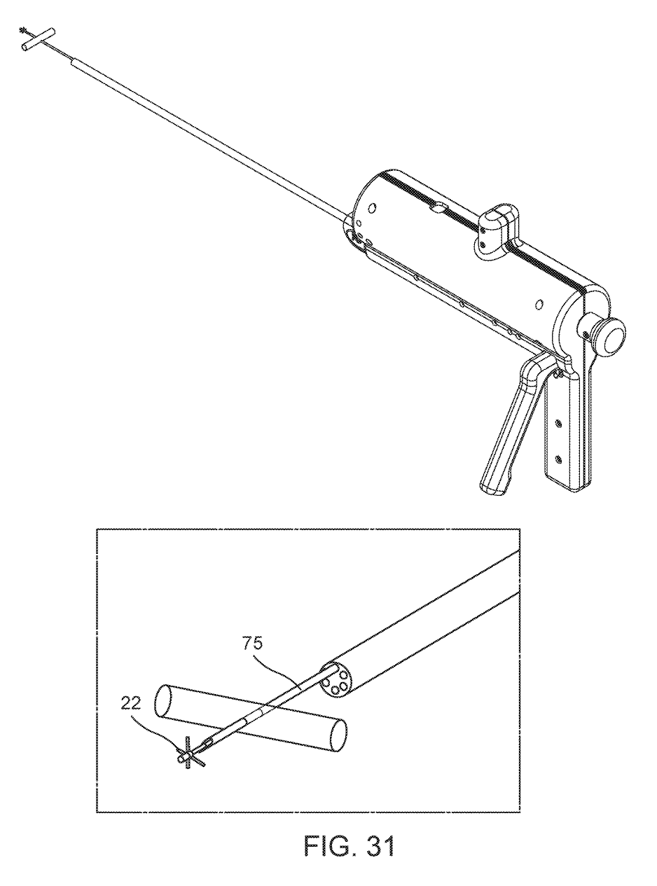

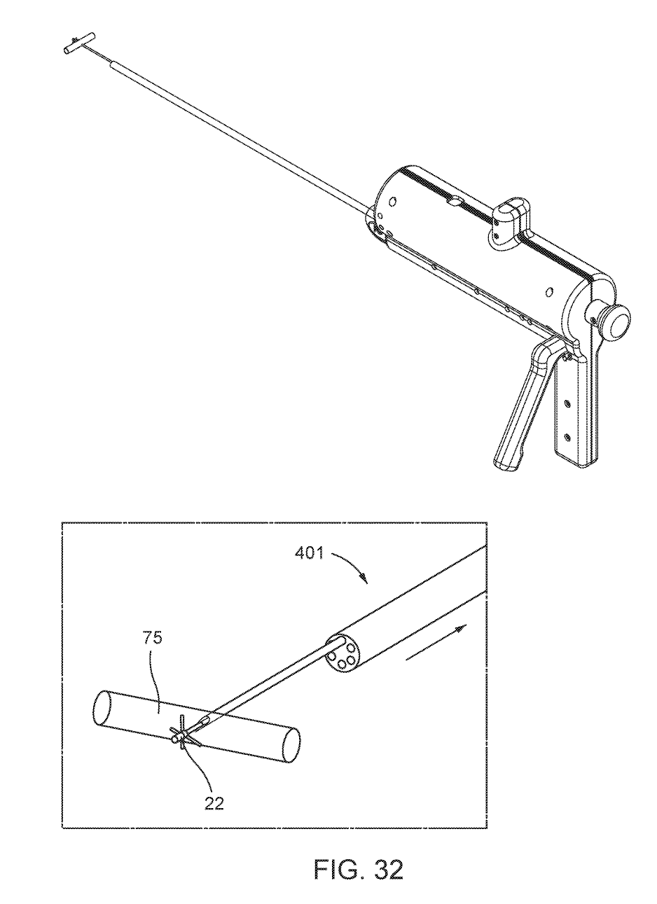

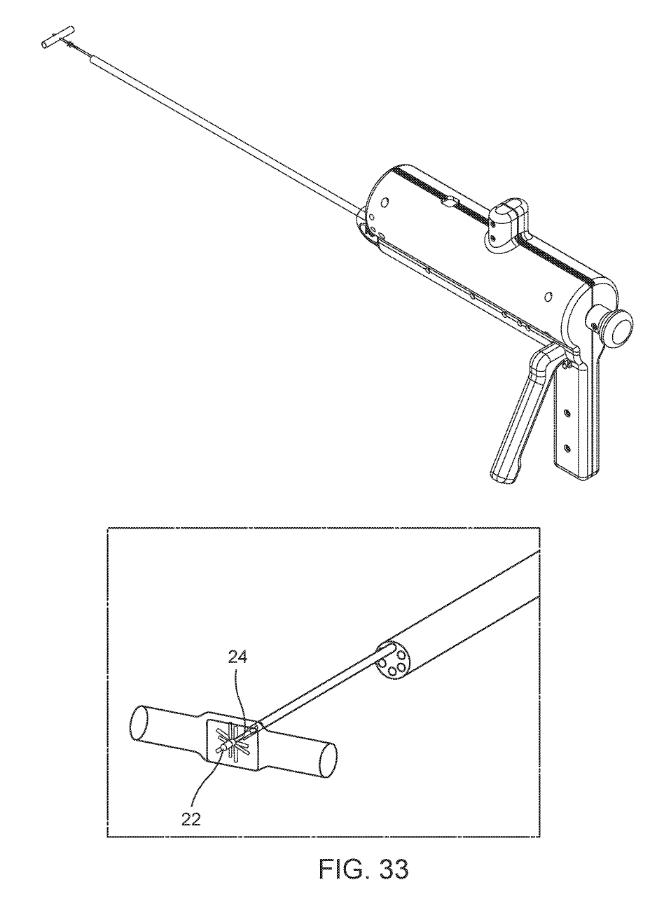

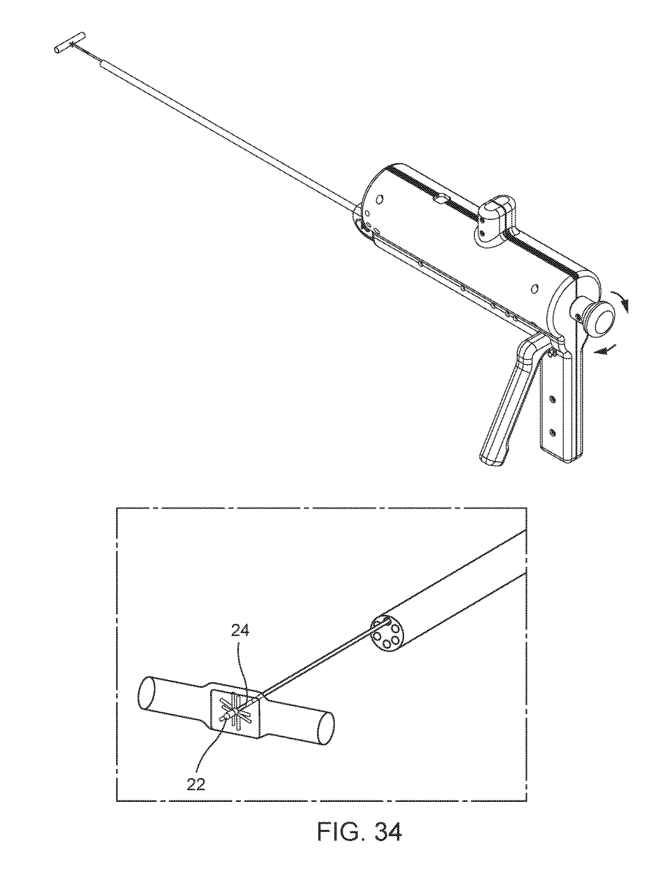

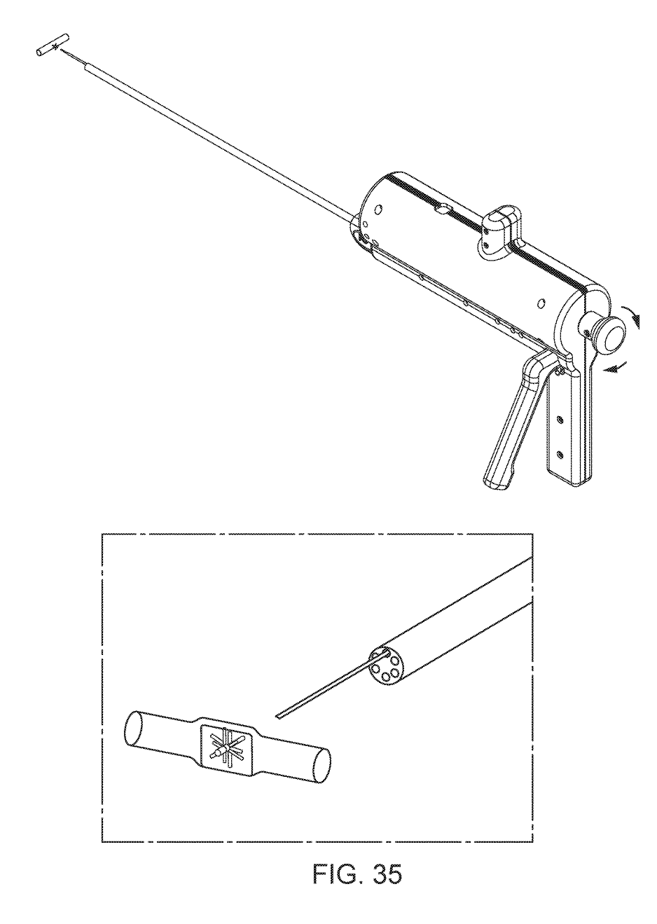

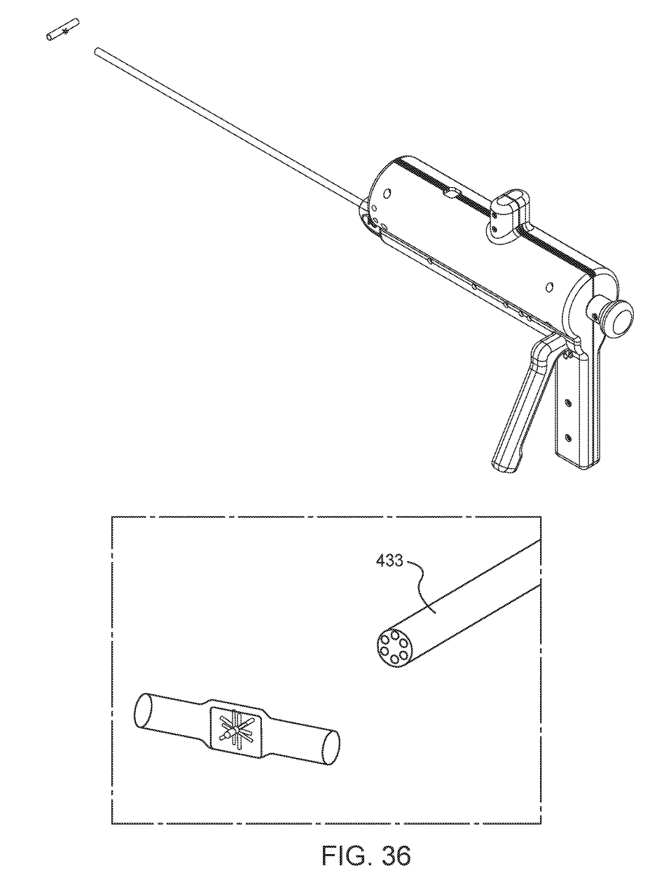

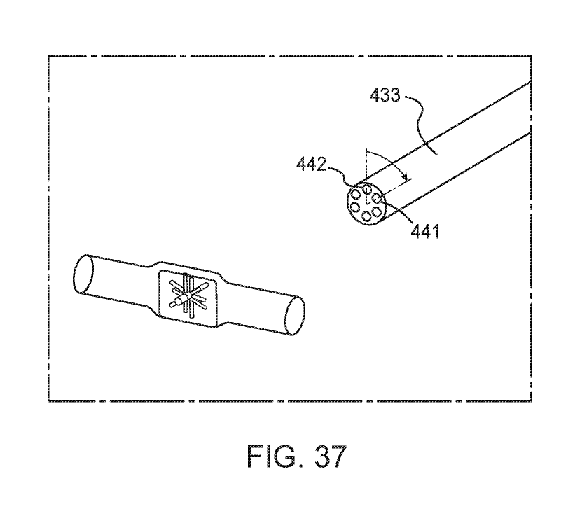

FIG. 28 shows a laparoscopic device 401 and an enlargement of the distal end of the laparoscopic probe with six chambers 441-446. As shown in FIG. 29, a needle 413 for deploying a first occluder is pushed out of a first chamber 441 when the knob 466 on the handle 471 is pushed in. The needle 413 can be positioned at the desired site for occlusion and is then inserted in and through the at least partially hollow structure 77 to be occluded, as shown in FIG. 30. As shown in FIG. 31, a distal occlusion element 22 is deployed from the needle 413 via an occluder pusher (not shown). In FIG. 32, the laparoscopic device 401 is pulled back until the distal occlusion element 22 is in close proximity to or touches the hollow structure 77. In FIG. 33, the proximal occlusion element 24 is pushed forward, resulting in the proximal occlusion element 24 locking to the distal occlusion element 22. In FIGS. 34 and 35, the knob 466 is rotated to release the locking mechanism (not shown, but substantially similar to the other locking mechanisms described herein) from the deployed occluder. In FIG. 36, the needle 413 is pulled back into the barrel 433. In FIG. 37, the barrel 433 is revolved to its next position, allowing the occluder delivery process to continue with another occluder pre-loaded into the chamber 442.

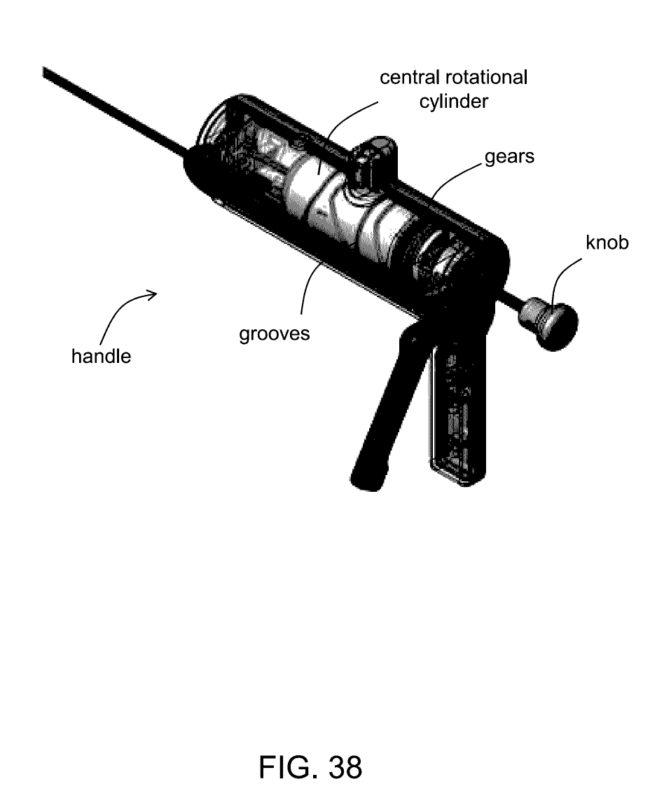

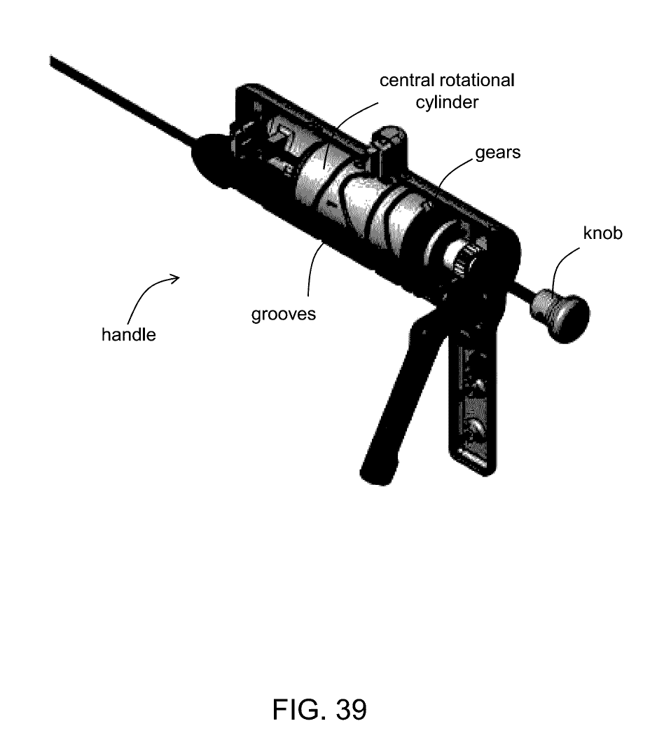

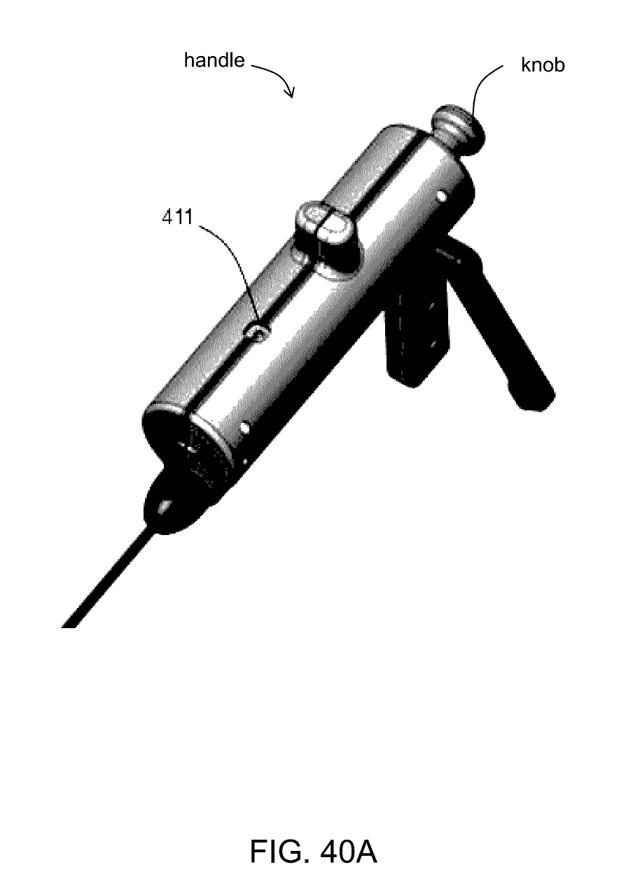

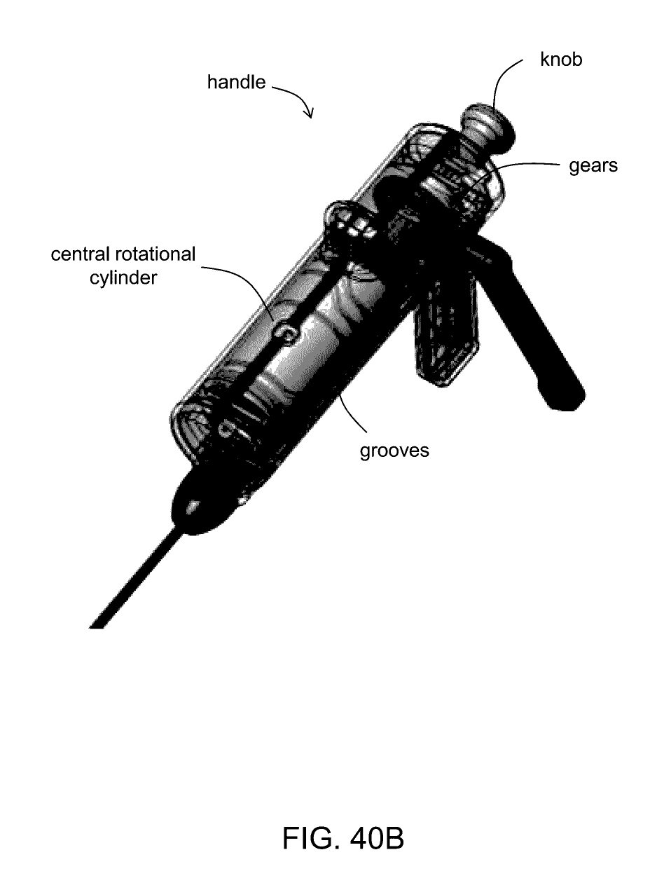

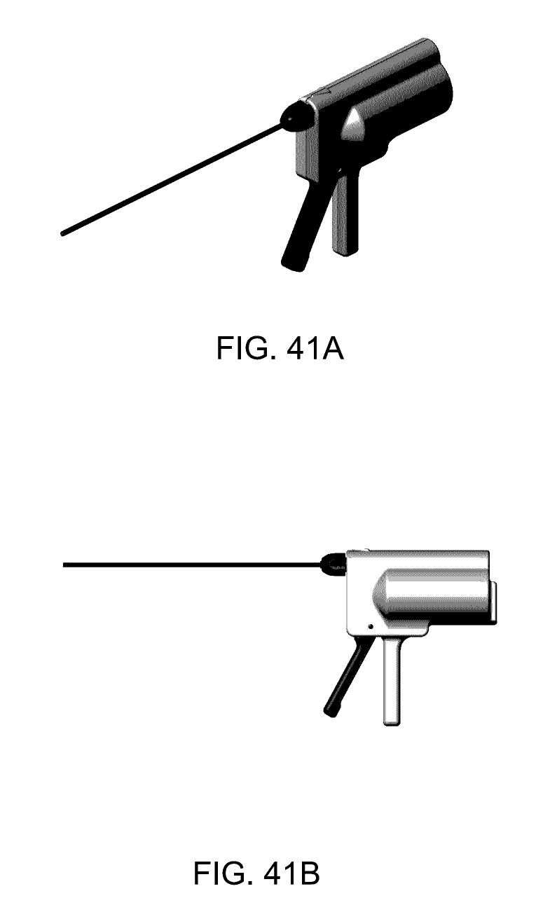

FIGS. 38-41 show detailed views of the handle, which is used to actuate the delivery steps shown in FIGS. 28-37. The actuator consists of a ratcheting device and a central cylinder with grooves. As the handle is depressed, the cylinder rotates, and actuates the various wires and rods, and needles and occluder elements to position and deploy the occluders. The central cylinder has a series of grooves cut into it, as the cylinder rotates, it pushes and actuates pins that ride inside the grooves, similar to a record player, and actuates the delivery of the needle out of the cannula, as well as actuates the various deployment steps of the occluder element. Each squeeze of the handle actuates a rotation of the central cylinder which moves pins attached to the various tubes, guide wires, needles and pushrods that then activate a corresponding deployment step of the occluder. The handle of the delivery device may include a window 411 (shown in FIG. 40A) indicating the number of occluders deployed or remaining. In yet another embodiment of the device, automatic cocking of the delivery device in a new needle is automatically achieved by actuating the handle of the delivery device. FIG. 41A and FIG. 41B. show an embodiment of a more compact multi-occluder delivery device, where the activation handle has been shifted to the front of the device.

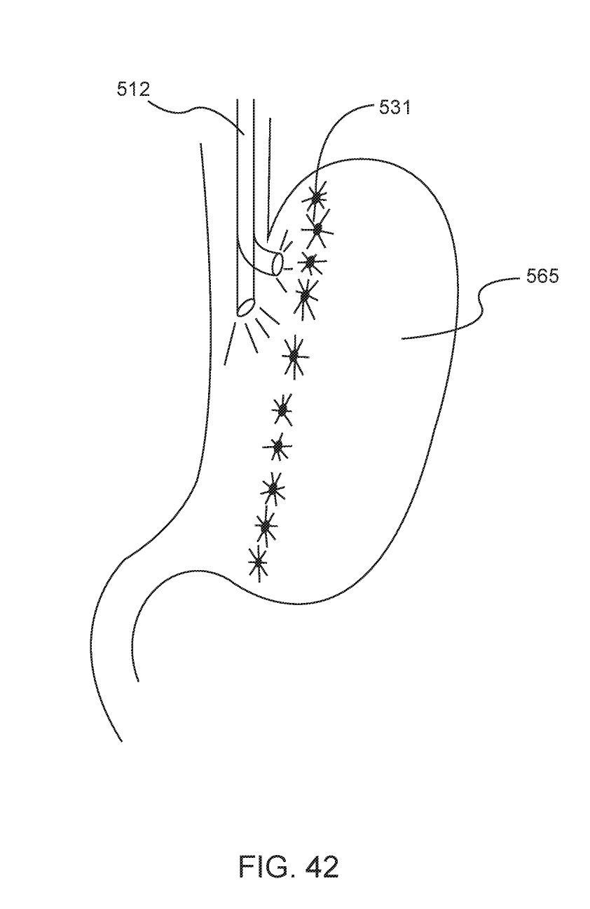

In certain embodiments, the multiple-occluder delivery device can be used for bariatric surgery. Occluders can be deployed to a patient's stomach 565 to create a gastric sleeve to reduce the size of the stomach. As shown in FIG. 42, occluders of the present invention can be delivered to a stomach percutaneously or using a laparoscopic device. The procedure may involve inflating the stomach with gas and illuminating with endoscopy or fluoroscopy. An endoscope 512 may be delivered to the stomach through the esophagus. Local anesthetic may be used. A multiple-occluder delivery device is inserted percutaneously or via laparoscopy to deliver a first occluder 531 by penetrating both walls of the stomach and deploying the occluder 531 to hold the two walls together. Delivery of the occluder is substantially the same as occluder delivery described throughout the disclosure. Subsequent occluders are delivered to reduce the effective size of the stomach 565 to a smaller pouch.

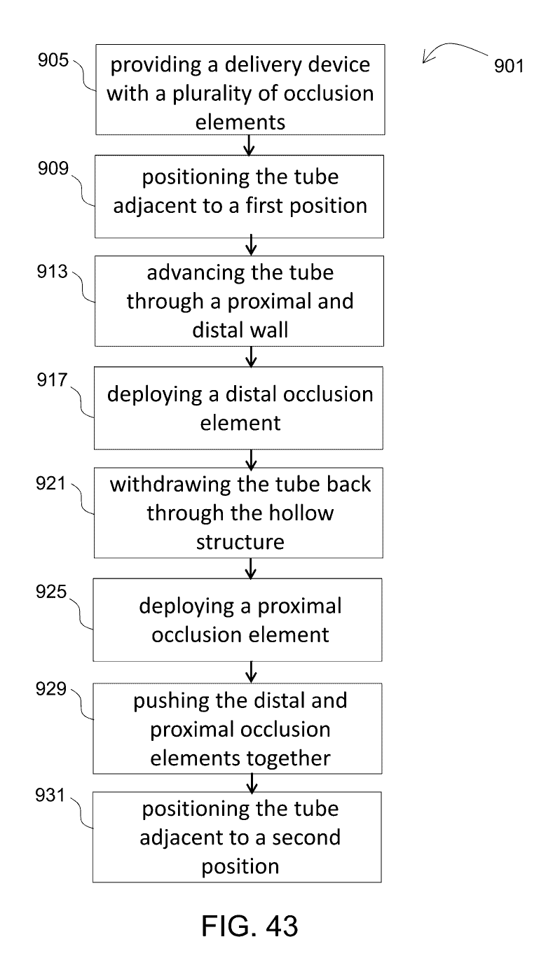

FIG. 43 shows the steps of a method 901 for occluding a hollow structure, according to the present invention. The method involves providing 905 a multiple-occluder delivery device containing a plurality of occlusion elements. The device includes a hollow tube, in which the occlusion elements may be serially arranged. The tube is positioned 909 adjacent to a first position on a hollow structure to be occluded. Next, the method involves advancing 913 the tube through a proximal wall and a distal wall of the hollow structure to be occluded. Once the tube has been pushed through the hollow structure, a distal occlusion element is deployed 917 from the tube. The next step involves withdrawing 921 the tube back through the hollow structure, and deploying 925 a proximal occlusion element from the tube on the near side of the structure. The proximal and distal elements are pushed 929 together to clamp the hollow structure, thereby occluding the first position. Next, the method involves positioning 931 the tube adjacent to a second position on the hollow structure. Steps 913-929 are repeated at the second position. The steps can be repeated as necessary, depending on the number of occluders needed for a particular procedure.

In certain embodiments of the method of delivery, the needle is inserted at an angle to the element to be occluded, or tissue to be clamped, to minimize the footprint of the occluder transverse to the tubular structure to be occluded or tissue to be clamped. Deployment of the occluder element occurs at an angle smaller than 90 degrees to the tubular structure, or tissues to be occluded or clamped. The occluder may be a one- or a two-part occluder. By deploying the occluder at an angle relative to the vessel, in some instances where the vertical connection point of the occluder is parallel to the vessel, the effective perpendicular protrusion of the occluder relative to the vessel is minimized.

### Pushing Mechanism

Throughout the disclosure, delivery devices are described that include arrangements of occluder elements disposed within a tube. In a typical arrangement of a serial delivery device, several proximal and distal occluder elements are lined up within the tube in an alternating fashion. In that way, a distal occluder element can be deployed out of the tube, followed by a proximal occluder element, which locks together with the distal occluder element to occlude a structure. The delivery device can then occlude another location or the same location by deploying the next distal occluder element, followed by the next proximal occluder element, and so on. The device is thus configured to make several occlusions without needing to be withdrawn from the patient and without needing to reload the device.

In some embodiments, each occluder is designed to push the occluder in front of it (and to be pushed by the occluder behind it). There are several ways to achieve that pushing action, as described herein. In a preferred embodiment, an occlusion element made of a shape-memory material such as Nitinol is shape set to achieve a particular geometry that allows the distal end of one occlusion element to firmly contact the proximal end of the element in front of it in the delivery device.

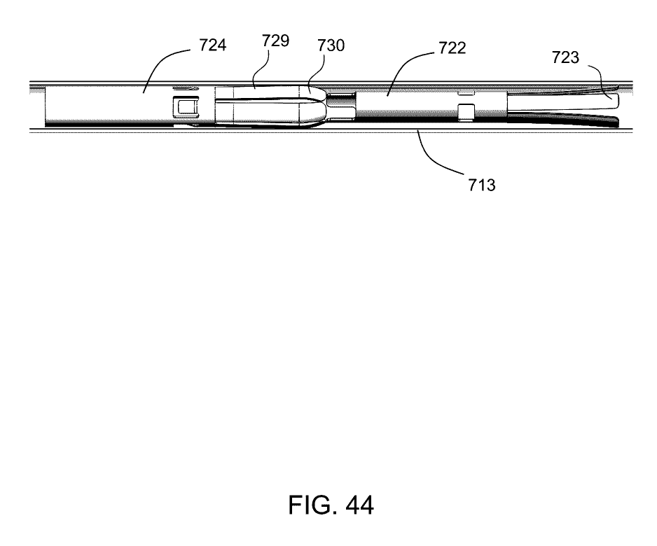

FIG. 44 shows two exemplary occlusion elements disposed within a delivery tube 713. In the configuration shown, the elements would be pushed out of the delivery tube 713 from left to right. The rear occlusion element is the proximal occlusion element 724 and the forward occlusion element is the distal occlusion element 722. Because the cylindrical section of the distal occlusion element 722 has a narrower diameter than that of the proximal occlusion element 724, the proximal occlusion element 724 includes fingers 729 with bent finger tips 730 that curve inward in order to contact the proximal end of the distal occlusion element 722. In that manner, even though the two elements have different diameters, they can still contact each other and push each other out of the tube 713.

Likewise, the distal occlusion element 722 is configured with widened finger tips 723, which allow the distal occlusion element 722 to make contact with the proximal occlusion element in front of it (not shown).

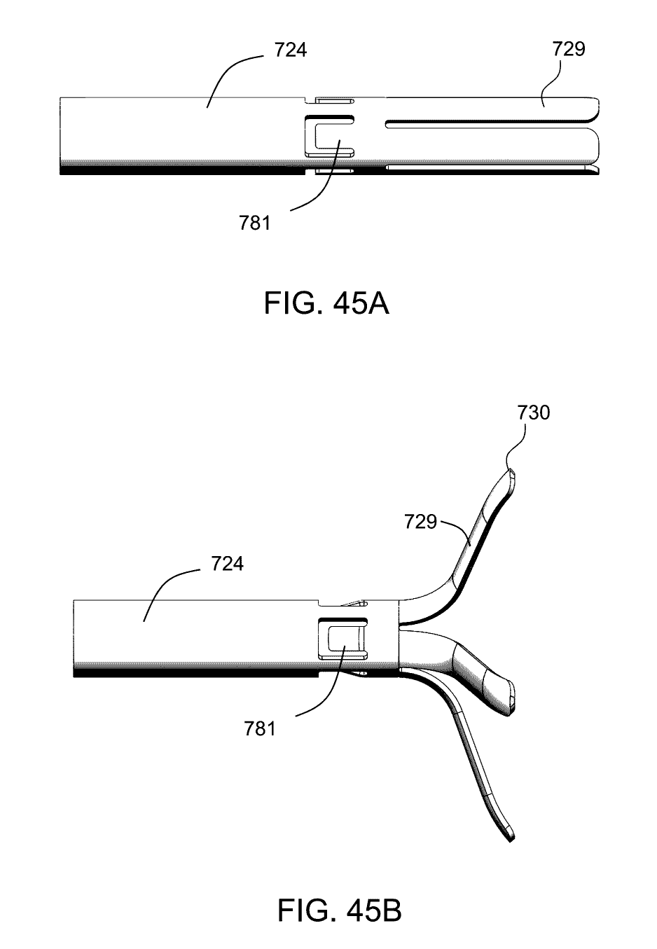

FIG. 45A shows a proximal occlusion element 724 following laser cutting. The occlusion clip 724 can be cut from a Nitinol cylinder. Slits are cut extending up a portion of the cylinder to define the plurality of fingers 729. Notches are cut about halfway down the cylinder to define tangs 781 which will make up part of the mechanism that locks the distal occluder and proximal occluder together during use. That locking mechanism is described in greater detail below, as well as in co-pending application Ser. No. 14/639,814, filed Mar. 5, 2015 (published as US 2015-0173765), which is incorporated by reference herein in its entirety.

Following laser cutting, as shown in FIG. 45B, the Nitinol is shape set to achieve the particular features of the tangs 781 and fingers 729. The tangs are bent inwards so as to connect with the corresponding windows of another occluder element (not shown), as described in greater detail below. The tips 730 of the fingers are shape set to be slightly bent inwards to be able to connect with the outer diameter of another occluder element (not shown) while they are both in the delivery tube or needle.

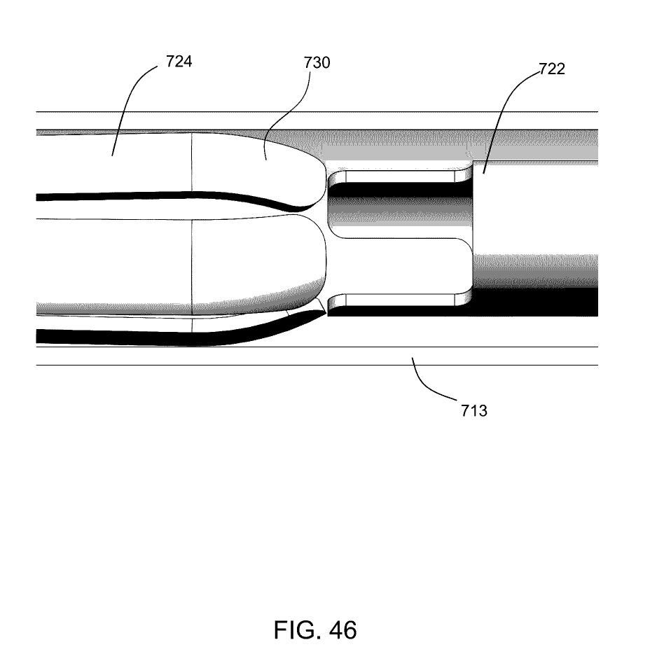

FIG. 46 shows a close-up view of the orientation of two occlusion elements within a delivery tube 713. The proximal element 724 has a larger diameter than the distal element 722, which allows the proximal element 724 to advance axially over the distal element when they are deployed. But when inside the delivery tube 713, the bent finger tips 730 of the proximal element 724 connect with the back end of the distal element 722, allowing the proximal occlusion element 724 to push the distal occlusion element 722 forward.

### Mechanism for Locking Proximal and Distal Occluder Elements

The proximal and distal occluder elements (also known as proximal and distal clips) are configured to lock together with a locking mechanism. The locking mechanism may include sets of windows and tangs that fit together. For example, the distal clip can be laser cut with one or more windows around the circumference of the hollow tubular section of the clip. The clip can include one window or several windows. The corresponding proximal occlusion element can be cut to include one or more tangs, each tang configured to snag into a window, thereby locking the two occlusion elements together.

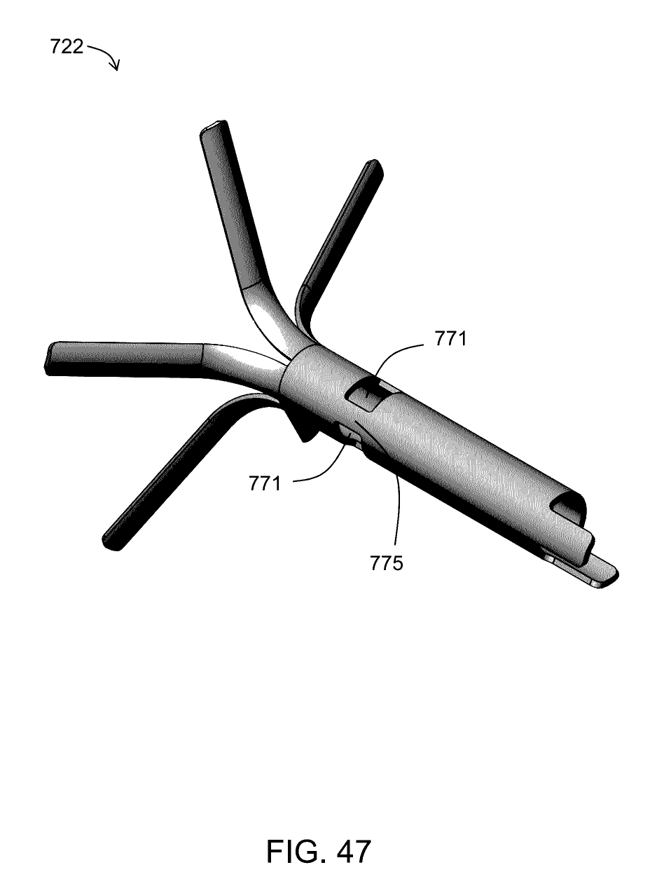

One concern with a tang-and-window mechanism is that the two occlusion elements must align so that the tang enters the window. The tangs and windows of the present invention are designed in such a way that they snag together regardless of the angular orientation of the occlusion elements. That design feature is known as “angular relation indifference,” and is illustrated in greater detail in FIGS. 47-55.

FIG. 47 shows a distal occlusion element 722 with three windows 771 (only two visible) spaced evenly around the circumference of the occlusion element 722, located proximal to the expandable fingers. Each window 771 is separated by a window frame element 775. In the embodiment shown, each of the three windows 771 occupies approximately 80 degrees of the circumference of the tube, and each window frame element 775 occupies approximately 40 degrees of the circumference of the tube. Those skilled in the art will appreciate that other arrangements and sizes of windows are possible that will still achieve the angular relation indifference configuration taught by the present disclosure.

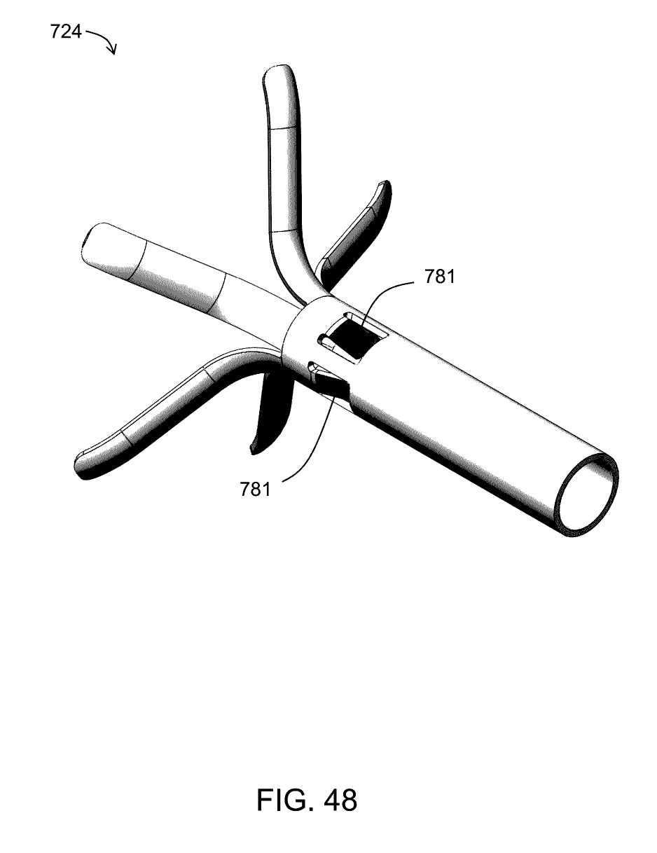

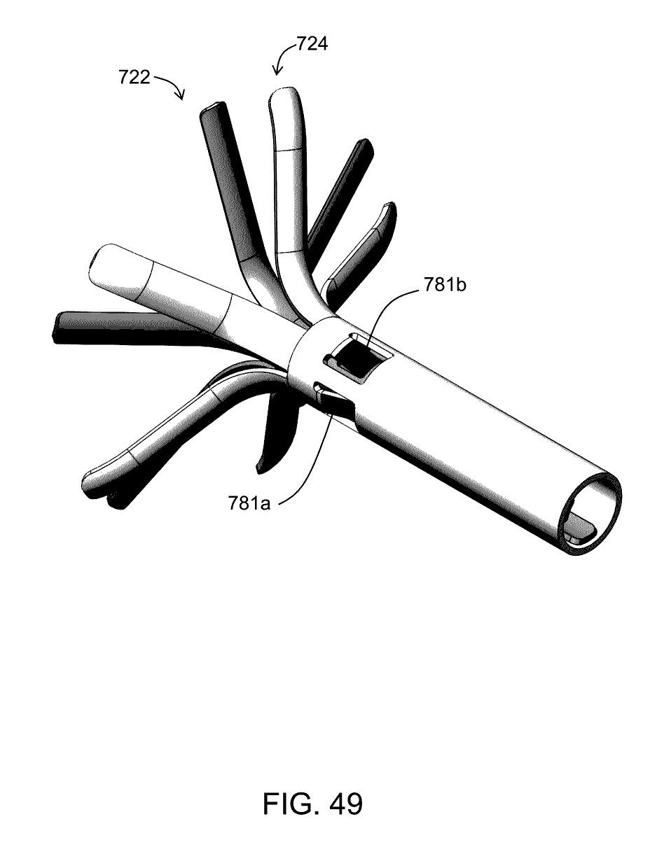

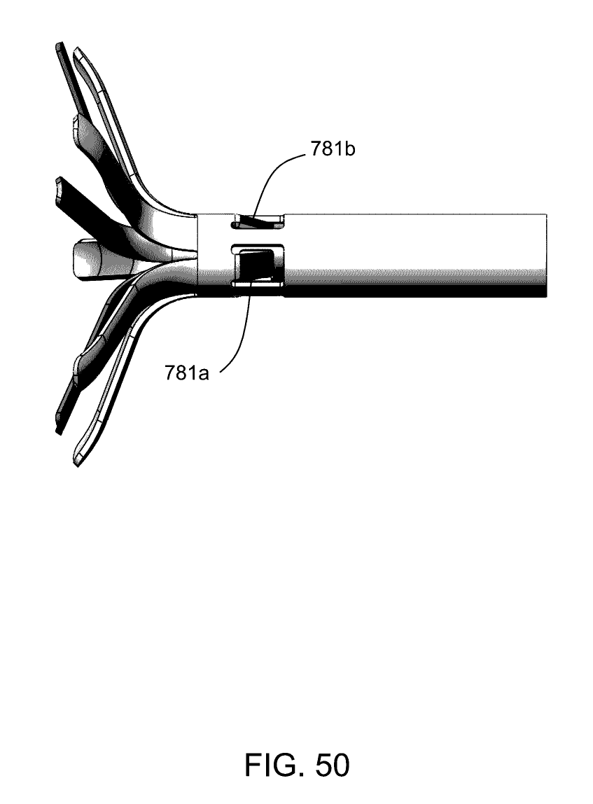

FIG. 48 shows a proximal occlusion element 724 with four inwardly-projecting tangs 781. Only two of the tangs 781 are visible in the drawing. Despite the different numbers of windows 771 and tangs 781, FIGS. 49-55 show that the distal occlusion element 722 and the proximal occlusion element 724 will lock together, regardless of their angular orientation.

FIG. 49 shows the proximal occlusion element 724 and the distal occlusion element 722 locked together. The distal occlusion element 722 has a smaller diameter than the proximal occlusion element 724 and fits within the hollow tube of the proximal occlusion element 724. The two visible tangs 781a and 781b are both projecting inwardly into the corresponding windows 771 of the distal occlusion element 722, thereby locking them in place. FIG. 50 shows a side view to more clearly show the inwardly projecting tangs 781a and 781b locked in place with respect to the windows 771.

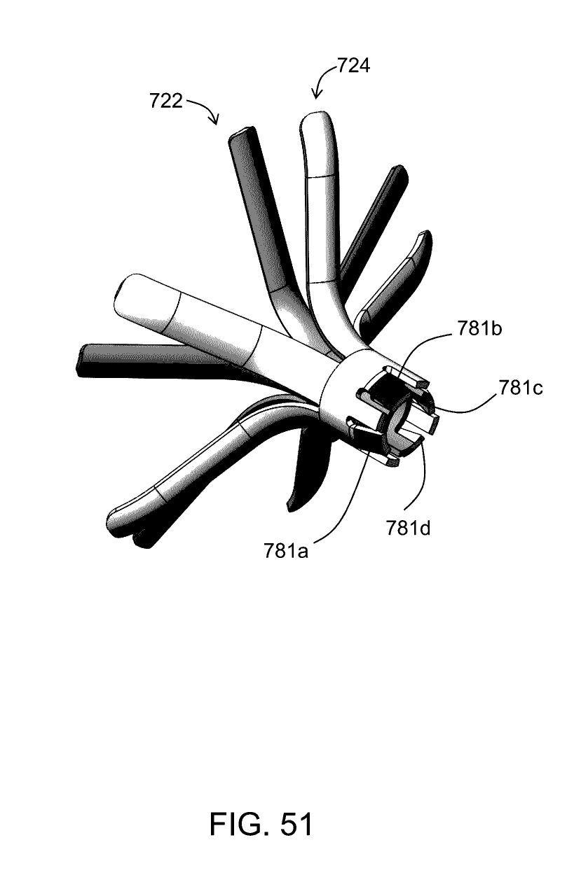

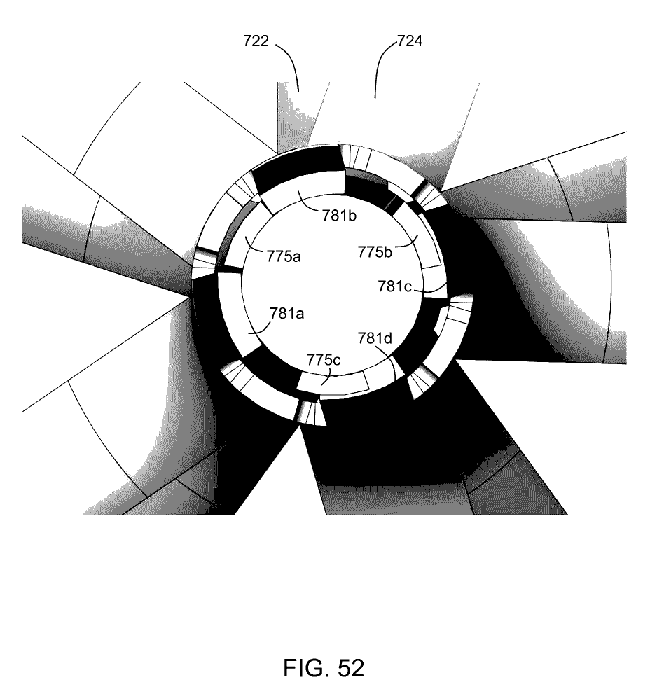

FIG. 51 shows a sectioned view of the two occlusion elements locked together. Due to the different configurations of the tangs 781 and windows 771 (i.e., there are four tangs 781a-d but only three windows 771a-c), tangs 781c and 781d do not project into the windows 771 in the orientation shown. However, regardless of orientation of the two occlusion elements, at least one (and as many as two) of the four tangs will always be locked in place. That angular relation indifference is shown more clearly in FIGS. 52-55.

FIG. 52 shows a close-up cross-section view of the two occlusion elements locked together. In this orientation, tangs 781a is locked in place in the window 771 defined by window frame elements 775a and 775b; and tang 781b is locked in place in the window 771 defined by window frame elements 775a and 775c. Tangs 781c and 781d are not projecting into windows, but are instead contacting window frame elements 775b and 775c, respectively.

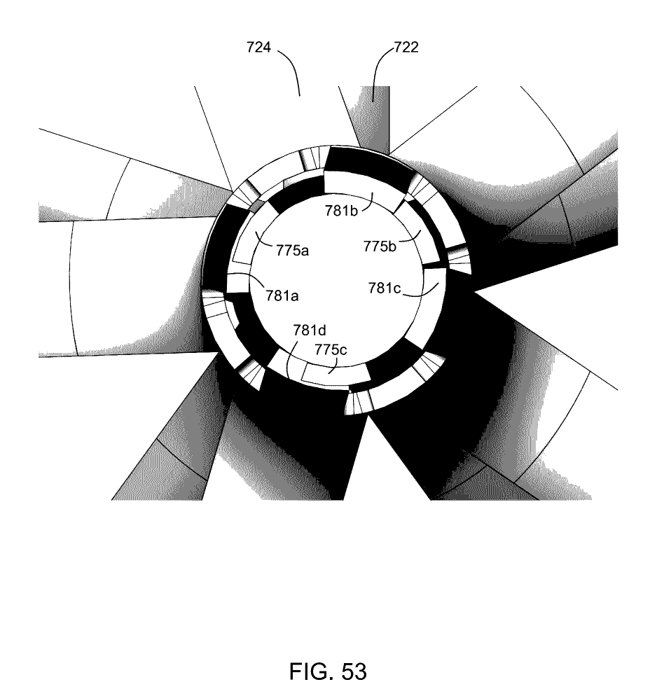

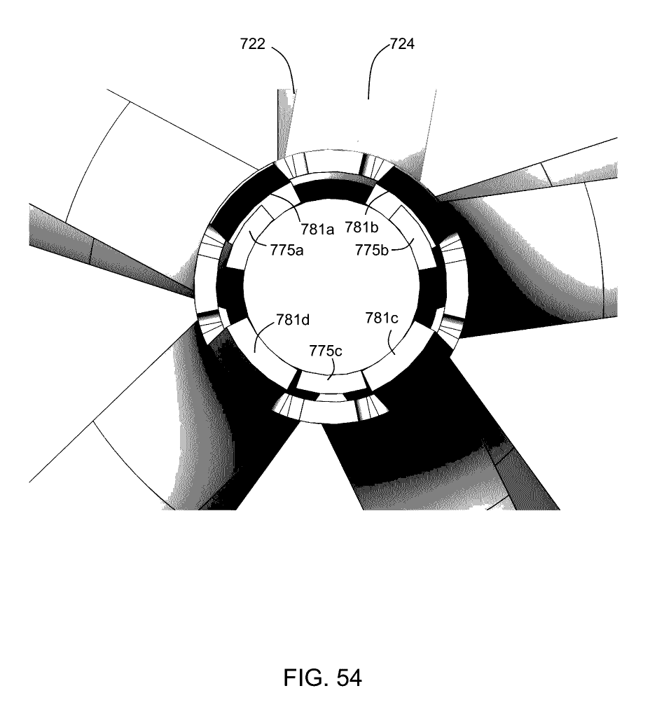

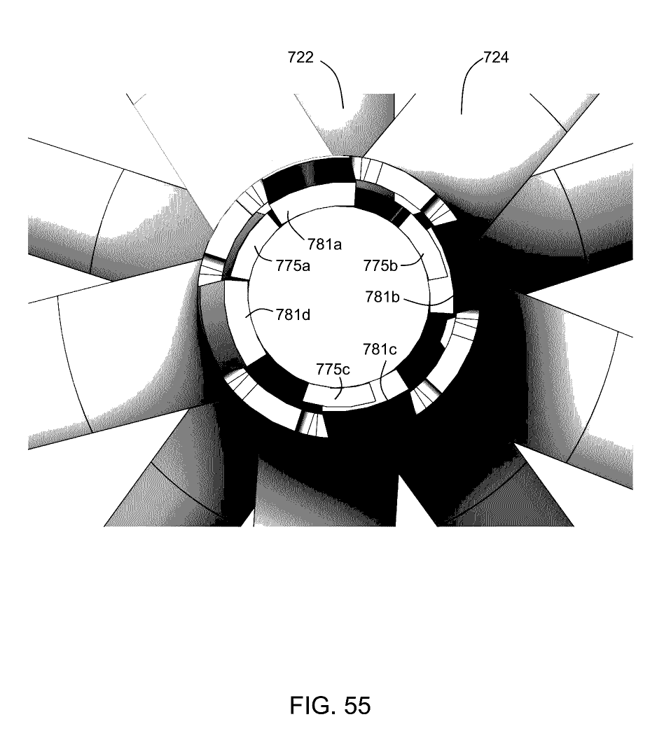

FIG. 53 shows a slightly different orientation between the two elements. This orientation is rotated clockwise from the orientation shown in FIG. 52. In FIG. 53, tangs 781b and 781c project into windows 771 defined by window frame elements 775a and 775b, and 775b and 775c, respectively. Tangs 781a and 781d are in contact with window frame elements 775a and 775c, respectively. FIGS. 54 and 55 show two other orientations, with the proximal occlusion element 724 turned clockwise with respect to the preceding orientation. Regardless of how many degrees the proximal occlusion element 724 is offset from the distal occlusion element 722 (from 0-360 degrees), in any orientation there will always be at least one, and as many as two, of the tangs locked into one of the windows, due to the angular relation indifference configuration.

In some embodiments, the tangs and windows are configured to connect together to orient the fingers of one occlusion element with the fingers of the other occlusion element. For example, the windows may be configured to receive the tangs and shift them into the desired position, thereby orienting the two occlusion elements in a certain way. It may be desirable, for example, for the fingers of a distal occluder to be offset with respect to the proximal occluder to achieve an interdigitating configuration. The benefits of interdigitation have been explained in detail above. In other embodiments, it may be desirable for the fingers to align so as to compress the structure between aligned fingers. Other alignments may be preferable as well, including partially offset fingers.

### Mechanism for Locking Distal Occlusion Element and Locking Rod

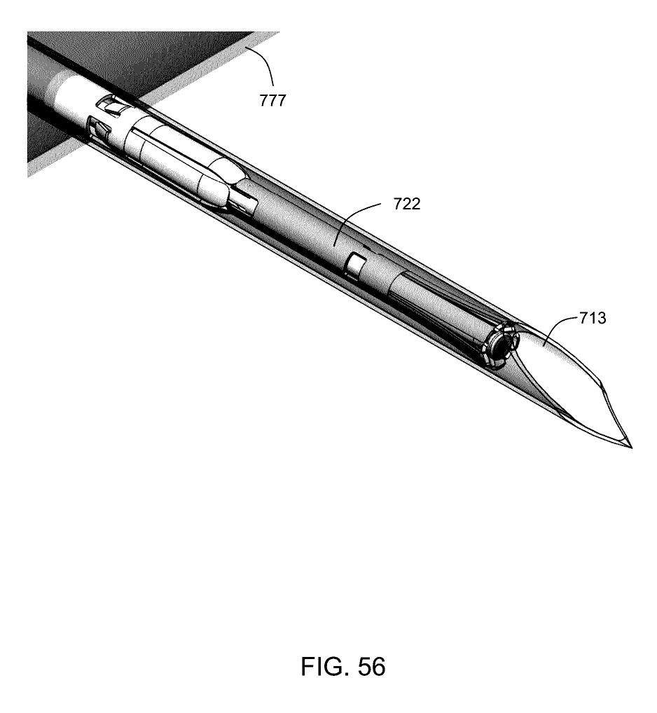

As explained above, devices of the invention include a locking mechanism for holding the distal occlusion element in place after it has been deployed from the delivery tube. Additional details of one embodiment of the locking mechanism are shown in FIGS. 56-63.

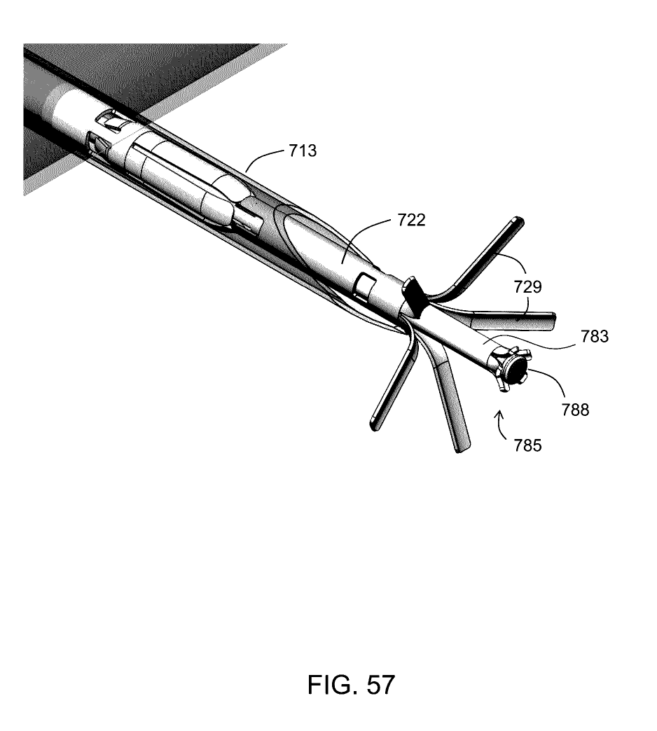

FIG. 56 shows a distal occlusion element 722 ready to be deployed from delivery tube 713. The delivery tube 713 has already been advanced through two walls of the hollow structure 777. In FIG. 57 distal occlusion element 722 has been pushed out of delivery tube 713 and its fingers 729 have expanded to their diametrically-expanded configuration. The locking sheath 783 surrounds the locking rod (not visible). The locking rod includes the locking tip 788. The locking tip 788 is in its pulled-back position, causing the locking sheath expansion region 785 to assume its expanded configuration.

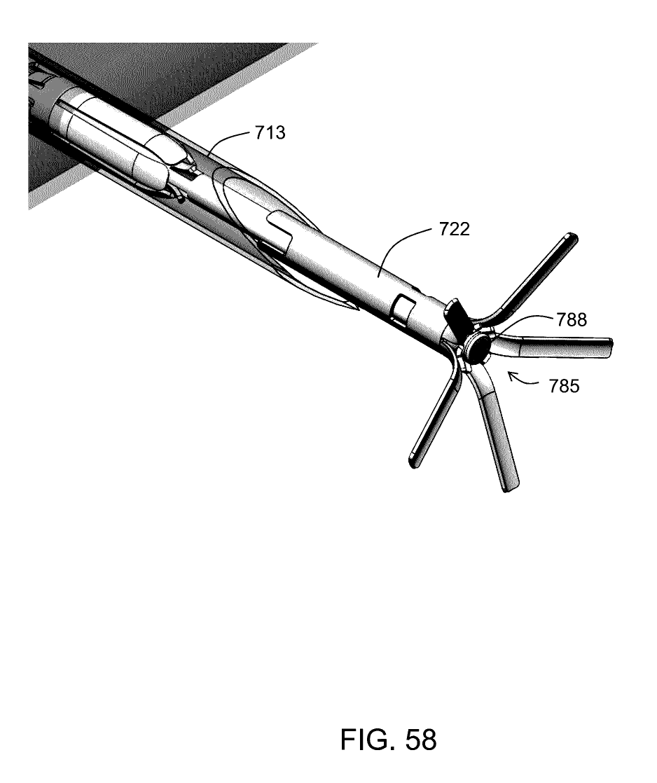

FIG. 58 shows the locking rod and locking sheath 783 pulled proximally, so that the locking sheath expansion region 785 contacts the distal occlusion element 722. Because the locking tip 788 has caused locking sheath expansion region 785 to expand, the sheath 783 cannot be retracted any further through the distal occlusion member 722. The locking sheath expansion region 785, in its expanded state, is a greater diameter than the inner diameter of the hollow portion of the distal occlusion element 772. The distal occlusion element is thus still coupled to the device, even after being deployed out of the delivery tube 713. The proximal occlusion element 724 can now be pushed out of the delivery tube 713.

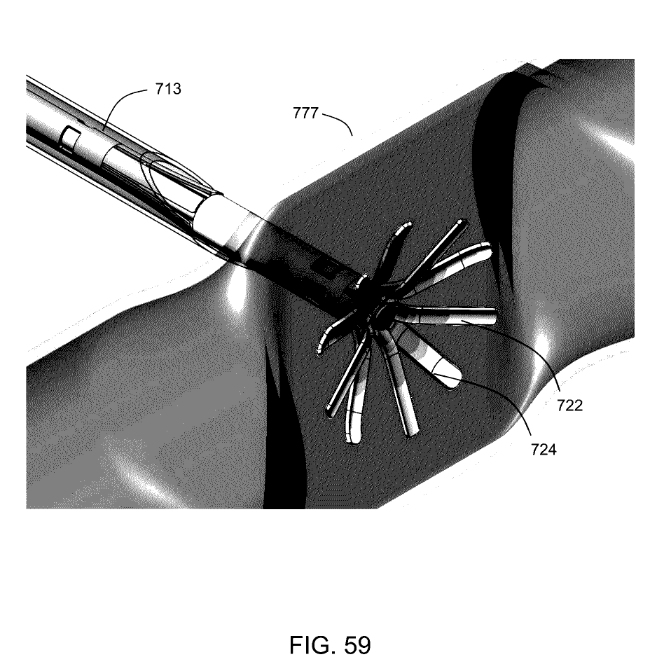

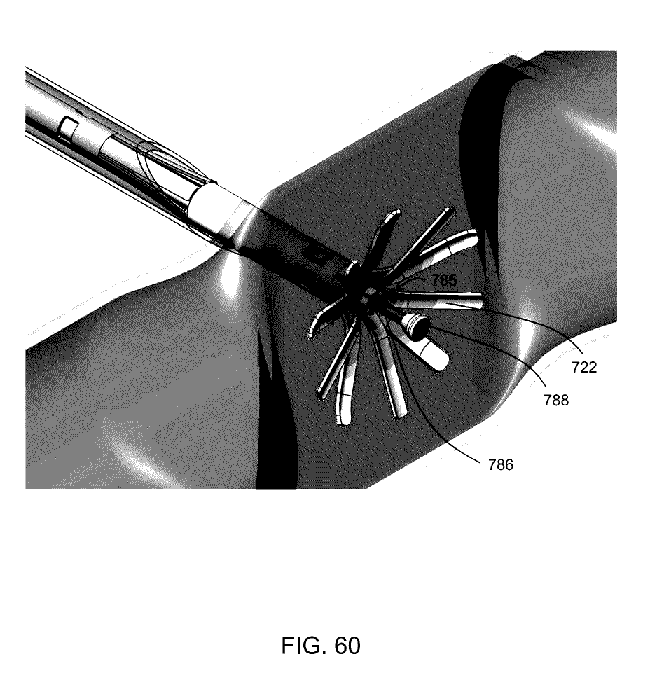

FIG. 59 shows distal occlusion element 722 remains locked in place with the locking mechanism. The proximal occlusion element 724 has been pushed out of the delivery tube 713 and is locked together with the distal occlusion element 722, thereby occluding a hollow structure 777. Once the occlusion elements 722 and 724 are locked together, the locking rod 786 can be detached from the distal occlusion element 722 so as to release the deployed occluder from the delivery tube 713. FIG. 60 shows the locking rod 786 advanced forward, causing the locking tip 788 to release the locking sheath expansion region 785 back into its non-expanded configuration. When it is not expanded, the locking sheath expansion region 785 has substantially the same diameter as the rest of the locking sheath 783. The entire locking sheath 783 can thus be withdrawn back through the hollow distal occlusion element 722.

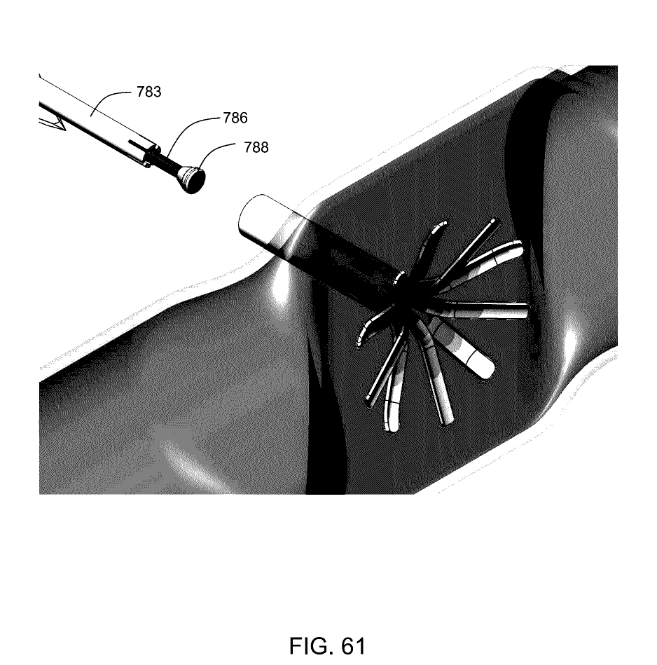

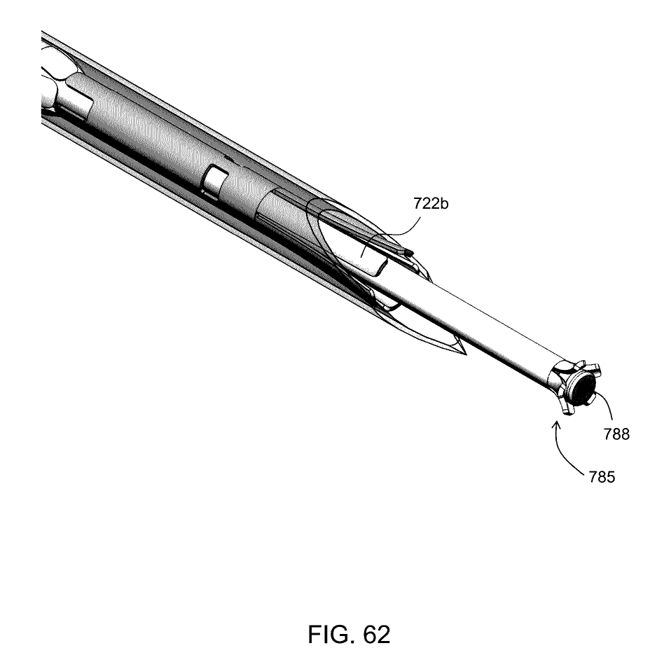

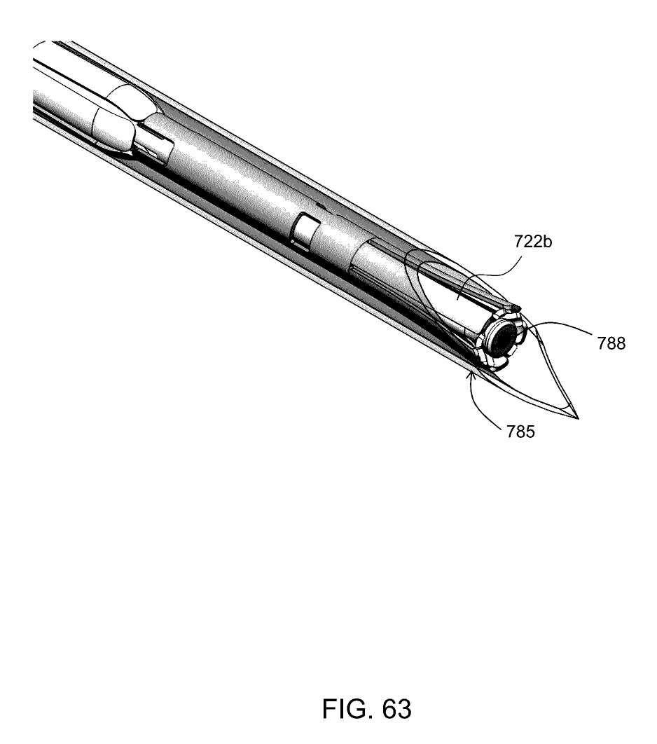

FIG. 61 shows the locking sheath 783, locking rod 786, and locking tip 788 withdrawn from the deployed occluder. In FIGS. 62 and 63, the locking tip 788 has once again engaged with and expanded the locking sheath expansion region 785, so that the locking mechanism is not withdrawn further back through the next distal occlusion element 722b. At this stage, the device is ready to deploy the next set of occlusion elements.

### Low-Profile Occluder

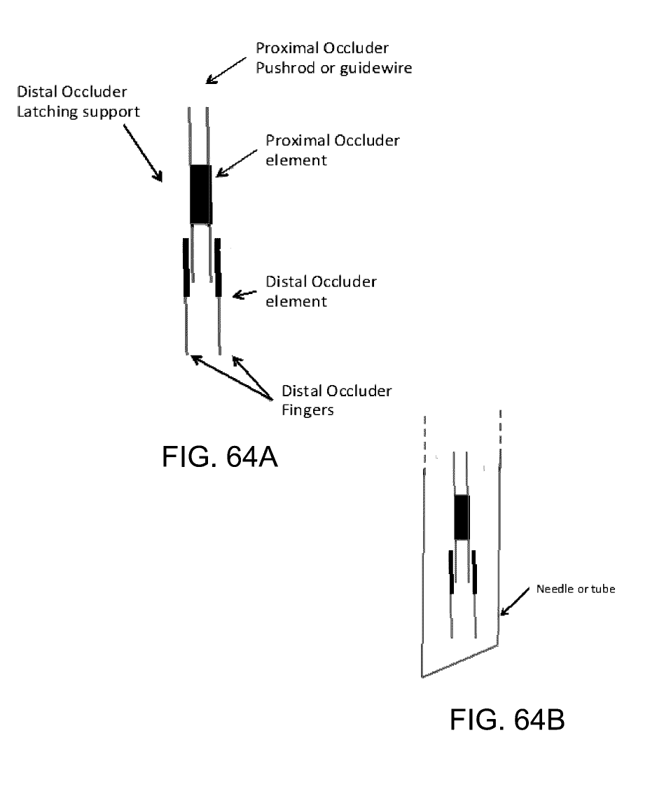

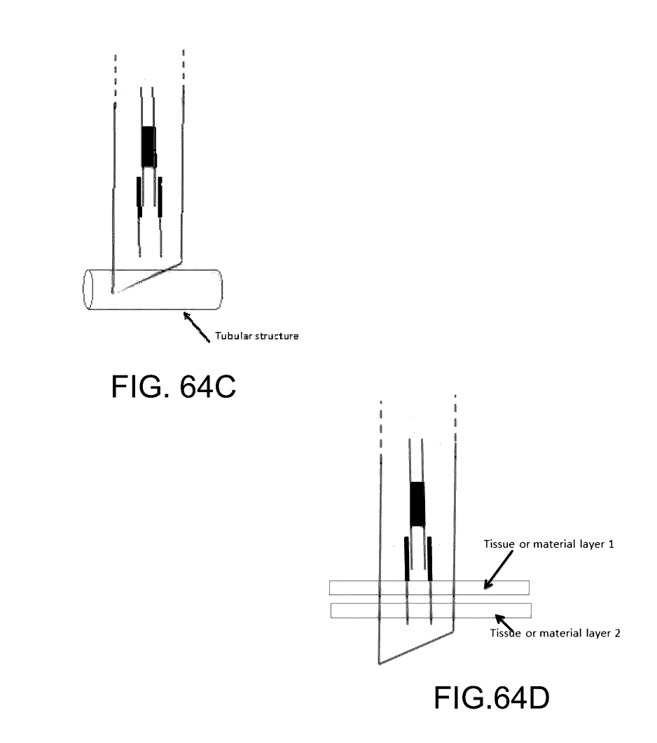

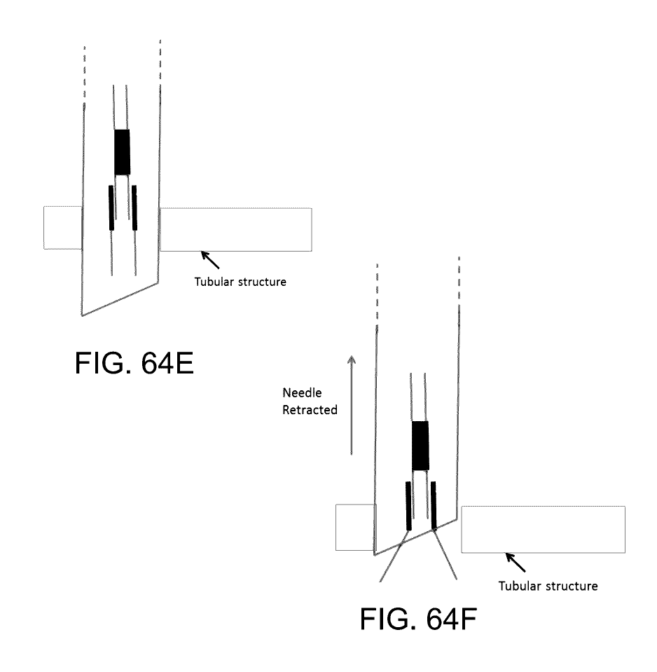

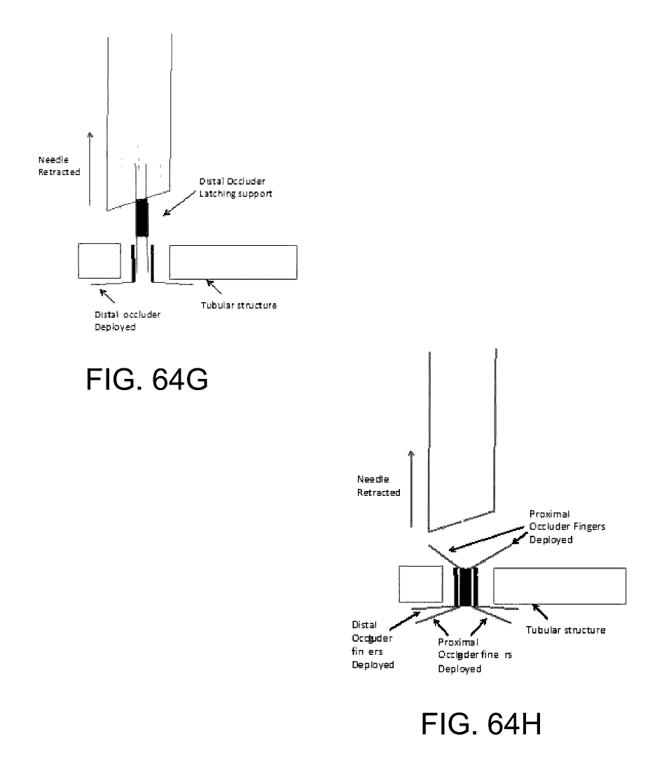

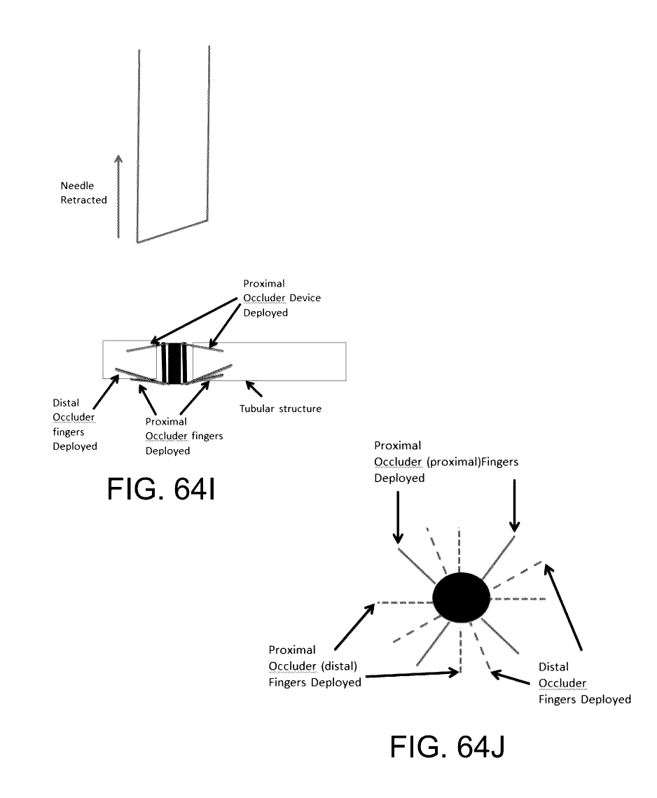

FIGS. 64A-I show a two-part occluder with a low vertical profile across the occlusion region, where the locking occurs outside of the overlap region of the occluders. Also, the profile of the two-part occluder is small, so that there is no locking region for connection to a locking rod, protruding from the occluder element.

FIG. 64A provides a cross-section showing proximal and distal occluder elements and supports that will be housed in needle or tubular structure. FIG. 64B is a cross-section showing proximal and distal occluder elements and supports contained in a needle or tube for delivery.

FIG. 64C shows delivery of a needle or tube across a tubular structure to be occluded, or tissue or material layers to be attached. FIG. 64D shows the deployment of a distal occluder across a tubular structure.

In FIG. 64E, the needle is retracted and distal occluder pushed forward to deploy, alternatively occluder may be held fixed, while needle is retracted and the fingers open up (show in FIG. 64F). In another embodiment, the occluder device is pushed out of the needle, by moving the distal occluder latching support towards the needle opening while the needle is held fixed. In another embodiment both the distal occluder latching support and the push rod are pushed downward towards the needle opening, to deploy the occlusion device. In FIG. 64F, the distal occluder fingers open up and deploy on the distal side of the tubular structure, while still being supported and held in place by latching support.

In FIG. 64G, the proximal occluder is pushed via a pusher rod, or guidewire, through distal occluder and deploys its own proximal and distal finger elements. Then push rod and distal occluder latching support are removed, leaving the occluded or clamped structure. FIG. 64H shows the device deployed, occluding the structure or clamping the layers of material. As can be seen in FIG. 64I, the two part occluder has a low profile, meaning, it does not protrude vertically beyond where the fingers contact the structure. In addition, more controllable occlusion can be achieved, because the clamping occurs from an occlusion element that is deployed on the distal side of the tubular structure, and a second element from the distal side, which sandwich together. The proximal occluder fingers are released outside of the proximity of the tubular structure, so the tubular structure does not interfere with the fingers opening correctly. The proximal fingers push down any intervening tissue to collapse it. As such, the clamping can occur across variable tissue thicknesses and provide a reliable and highly controllable operation. FIG. 64J is a top view showing the various fingers deployed for the proximal and distal occluders. Note the fingers may also overlap or partially overlap in some embodiments.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### Appendix

### A Novel, Simple, and Secure, Percutaneous Vessel Occluder for the Treatment of Varicose Veins

Background

Secure, permanent occlusion of the great and small saphenous veins, their tributaries, and perforators, is critical for the successful treatment of varicose veins. Current minimally invasive methods replacing surgery are all endoluminal, and involve heat (radio-frequency or laser), chemicals (sclerosants and glues) or a combination mechanical and chemical (MOCA). The objective of this study was to evaluate in a porcine model, the performance of a percutaneous delivery of the Amsel™ Vessel Occluder (AVO) utilizing ultrasound guidance. The AVO has previously received FDA pre-market 510(k) clearance for use in open surgical procedures for tubular structures ranging in diameter from 2-7 mm.

Methods:

The AVO, a novel mechanical occlusion clip similar to a transfixion suture, is delivered through an 18G hypodermic needle, which transfixes the targeted vessel. The AVO is subsequently expanded on either side of the vessel wall, collapsed and locked together to effect secure vascular occlusion. Under general anesthesia, the targeted vessels in five swine, weighing >60 kilograms, were identified and vessel size measured. Patency of the targeted vessels was confirmed on duplex ultrasound (DUS). Each animal provided multiple vessels for percutaneous AVO occlusion. Occlusion was confirmed by DUS and by direct examination of the occluded vessel after open surgical exploration.

Results:

30 vessel occlusions were performed percutaneously including the common and superficial femoral arteries and veins (n=25), the carotid artery (n=3) and the external jugular veins (n=2). Measured vessel sizes ranged from 1.8-12.7 mm. Following vessel transfixion, occlusion was achieved in less than 30 seconds. A second AVO, if necessary, was employed to completely occlude the targeted vessel where the vessel was larger than 7 mm diameter (n=2; external jugular vein 12.7 mm and carotid artery 7 mm), or where the initial AVO did not occlude the vessel due to non-transfixion (n=1). Post-occlusion surgical exposure confirmed that all targeted vessels were successfully occluded and demonstrated no evidence of injury to any of the adjacent structures.

Conclusions:

This study confirms that the AVO can be effectively delivered percutaneously in the porcine model to occlude blood vessels under ultrasound guidance. The AVO provides a mechanical means of permanent, secure vessel occlusion, similar to a transfixion suture, thus eliminating the problem of recanalization which may occur following thermal or chemical vessel occlusion methods. This method of permanent, percutaneous occlusion may be a useful, time-saving and cost-effective adjunct to current primary methods of treating reflux in the saphenous veins, their tributaries or perforators, for the treatment of symptomatic varicose veins. In addition, in the event of other treatment method failures (thermal or chemical) the AVO provides a simple alternative.

Introduction

Varicose veins and its late manifestations are common in the adult population, increasing in prevalence with increasing age. It is estimated that 23% of US adults, suffer from varicose veins and 6% from chronic venous disease. It is generally more common in women than men between the ages of 40-80 yrs, although this difference decreases with age. Untreated, varicose veins may eventually progress to severe chronic venous insufficiency with symptoms and other manifestations including lower extremity venous ulceration. The related health care costs associated with chronic venous disease approaches one billion dollars annually in the US.

Although the pathophysiology of chronic venous disease, with lower extremity venous valvular dysfunction has been known since Trendelenburg described his tests for VV, most of the care is still directed at treating varicose veins only when symptoms or complications occur. The standard of care in the US for the treatment of varicose veins until the turn of the 21^{st} century was surgery with high ligation and stripping of the saphenous vein with phlebectomy of the presenting varicosities. More recently, alternative treatments for varicose veins including endothermal ablation, with laser or radiofrequency, together with chemical treatments, sclerosants and chemical adhesives or a combination (MOCA), have replaced surgery as the standard of care. These procedures have brought the treatment of varicose veins out of the hospital surgical operating rooms and into the outpatient surgical clinics, reducing hospital costs for patient management and minimizing patient morbidity. This migration to an office-based procedure has also altered who performs the procedure

The underlying pathophysiological approach to the treatment of these patients has not changed and is aimed at preventing reflux in the lower extremity veins by occluding a long segment of the saphenous vein from the sapheno-femoral junction (SFJ) to just below the kneeand phlebectomy of the superficial varicosities.

In the early 1990's Claude Franceschi, described an alternative saphenous “sparing” approach to treat the venous reflux associated with varicose veins. This approach, CHIVA (Cure conservatrice et Hemodynamique de L′Issufsance veineuse en Ambulatoire or conservative hemodynamic cure for varicose veins) unlike the surgical and endovascular approaches does not eliminate all reflux saphenous vein, but directs the flow of the “refluxing” blood into the deep veins of the lower extremity. In theory this relieves the venous pressure the superficial veins of the extremity and eliminates the clinical consequences of varicose veins. Because of the combination of demanding ultrasonic expertise, and the necessary surgical skills required to execute this approach, CHIVA has not been widely adopted, particularly in the United States.

For both pathophysiological approaches secure, permanent occlusion of the targeted veins, which include the saphenous veins, their tributaries, and perforating veins, is critical for the successful treatment of varicose veins and venous insufficiency. One of the concerns with the chemical and adhesive techniques is “leakage” of the chemical into the deep system, especially at the sapheno-femoral junction (SFJ). Indeed, the VenaSeal protocol encourages compression of the great saphenous vein just below the SFJ. Permanent and percutaneous of the GSV at this location would be advantageous.

The objective of this study was to evaluate the safety and efficacy of a novel mechanical occlusion clip, the Amsel™ Vessel Occluder (AVO), delivered percutaneously with ultrasound guidance in the porcine model.

Materials and Methods:

Device Description:

The Amsel Occluder Clip, is preloaded in the 18G needle of the AVO delivery device (FIG. 1). The AVO is a mechanical occlusion clip with a length 7.95 mm after occlusion, that when deployed transfixes the target vessel while clamping it shut. The AVO consists of two “star” shaped compression elements and a titanium fine strut which connects and locks the compression elements together. The proximal element, which compresses the near wall of the blood vessel or a tubular structure, and distal element, which compresses the far wall of the vessel are made of shape memory metal (nitinol), which once deployed, assumes its designated configuration closing off the vessel (FIG. 2). The individual “arms” of the proximal occlusion component alternate with and interdigitate with the individual “arms” of the distal occlusion components (FIG. 1a FIG. 3).

The AVO clip and delivery device has received FDA 510 (k) clearance, similar to other metal non-transfixing clips (hemoclips) for the occlusion of blood vessels 2 mm-7 mm in diameter, as well as for other tubular structures such as the cystic duct or fallopian tube.

Methods and Technique

Five female domestic pigs (Sus scrofa domestica), each weighing more than 60 kg at time of implantation, underwent percutaneous occlusion of selected veins and arteries (N=30). The study was performed at the Lahav Institute of Animal Research, Israel, under the supervision of the veterinary surgeon, the scientific manager at this institute. The studies were approved by the Institute of National Animal Care and Use Committee, Israel. Each animal was allowed to acclimate for at least 3 days prior to surgery. On the day of surgery food was withdrawn and only water was allowed.

Prior to occlusion, the selected arteries and veins in the groins and neck were examined with Duplex ultrasound. Vessels were selected, measured for size, and patency confirmed with Doppler Ultrasound. Similar to that for any minimally invasive access device a small incision was made in the skin at the site of needle entry. The ultrasound transducer was held in one hand and the AVO in the other hand. Under ultrasound guidance the needle of the device is inserted into the targeted vessel. A small hole in the needle approximately 6 cms from the needle tip allows blood to escape confirming entry into the vessel lumen (Figurela). The pulsatility and color of the bloods helps to distinguish between artery and vein. The needle is passed through the vessel, transfixing the vessel. Once the vessel is transfixed, ultrasound guidance is not essential. The AVO clip is delivered with 4 consecutive simple manoeuvres on the delivery device: the distal occluding element is delivered, assumes its predetermined configuration and is gently pulled back onto the vessel confirmed by a slight resistance; the proximal occluding element is then delivered and the two elements locked together. Finally, the locked occluding elements are released, allowing the needle and delivery device to be withdrawn (FIG. 1a-d).

Study Design

Selected vessels (Table 1) were examined with Duplex ultrasound, measured and patency confirmed with Doppler flow. Three board certified interventional radiologists participated in this study. Following each percutaneous occlusion, Duplex examination of the occluded vessel was performed to confirm occlusion (FIG. 3). The vessel and the occlusion site were then surgically exposed and inspected for any bleeding into the surrounding tissues, or any injury to the adjacent structures FIG. 3. The occlusion of the targeted vessel was confirmed by inspection by an independent observer, photographed and the occluded vessel excised. For arteries, confirmation also included division of the vessel between the percutaneously inserted AVO and a distal hemostat and confirmation that the arterial segment proximal to the AVO was patent by incising the artery. Pulsatile bleeding from the incised artery confirmed patency.

The integrity of the AVO clip occlusion with holding pressure measurements, as well as the tissue response and healing of the implanted occluding AVO by both inspection and histo-pathological examination has been previously documented and reported following surgical vessel occlusion with the AVO.

Results

30 vessel occlusions with the AVO were performed percutaneously in 5 different pigs by 3 different interventional radiologists (IR1, IR2, IR3) (Table 1). The size of the vessel occluded, the number of clips required for occlusion and the success of the occlusion confirmed with Duplex ultrasound and surgical exposure, are shown in Table 1.

There were 10 proximal femoral artery and 8 proximal femoral vein occlusions (vessel size range: 3.6-7 mms), and 5 distal femoral arteries (superficial femoral arteries) and 1 distal superficial femoral vein (vessel size range: 2-3.5 mms) occlusions; 4 carotid artery occlusions (n=3: vessel size range 3.6-7 mms, n=1: vessel size 8.3 mms), one external jugular vein (vessel size:12.7 mms) and one tributary of the external jugular vein (vessel size: 9.5 mms) occlusions. It should be noted that all occlusions were achieved with a single AVO, except for the external jugular vein (vessel size 12.7 mms) in which 2 clips were required for complete occlusion.

The targeted vessel was not occluded on two occasions, one due to the clip being placed on the edge of the femoral vein and not centrally transfixing the vein) and the other, where one AVO was delivered outside the vessel into the underlying muscle. The IR recognizing the misplacement placed a second AVO to occlude the vessel. All other vessels were occluded with a single AVO clip.

In two femoral vein occlusions, where the AVO was not centrally placed, however, the veins were found to be completely occluded, with no Doppler flow. On surgical exposure, complete vessel occlusion was confirmed. However, the expanded occlusion elements of the AVO clip although not centrally placed had included some surrounding connective tissues to achieve complete occlusion of the vessel.

Despite the close proximity of the femoral vein, artery and nerve, in their tight neurovascular bundle covered by the muscle in the groin of the pig, each targeted vessel was occluded without impinging on the other vessel and or the accompanying nerve, and without injury to any of these adjacent structures. These occlusions in the pig are considerably more demanding than in the targeted veins for the treatment of varicose veins.

Discussion

This study confirms that the AVO can be effectively and safely delivered percutaneously under ultrasound guidance to accurately and securely occlude blood vessels in the porcine model. This represents a first in the ability to occlude blood vessels ranging from 2-12.7 mms directly through the skin via a fine hypodermic needle. The Amsel Occluder's novel design combines three key characteristics, bi-planar clamping that enables easy and controlled occlusion of blood vessels, transfixion, that provides anchoring similar to a surgically applied transfixion suture that secures the implant in place and eliminates slippage, and grasping with interweaving between occluding “arms”, which allows occlusion of both arteries and veins of different sizes and wall thickness, and ensures an area of hemostasis around the needle entry point (FIG. 3).

The Amsel Occluder and delivery device used in this procedure is of a prototype design that can easily be modified to optimize its performance depending on the circumstance. Although the AVO occluder is only cleared for patient use for vessels between 2-7 mms, we have successfully occluded vessels up to 9.5 mm with a single AVO clip (see Table 1). In larger vessels, placement of a second AVO clip has successfully occluded larger vessels. However, the size and length of the occluder used in this study can be easily be modified to match the vessel size, such that single clip vessel occlusion can easily be achieved.

The technique is simple, and is similar to other standard percutaneous interventional procedures. Our device involves minimal patient discomfort and recovery, with only a local injection of anesthetic at the site of percutaneous insertion. The entire procedural time is minimal, less than 30 seconds per occlusion, making such an occlusion method a very cost-effective adjunct to current primary methods of treating reflux in the saphenous veins, their tributaries or perforators. All three interventional radiologists participating in this study achieved great success and facility with minimal time spent in familiarizing themselves with the delivery device.

The possible clinical applications for the AVO in varicose veins are summarized in Table 2. Of the many advantages and opportunities in the application of such a mechanical, secure and permanent vessel closure device (Table 2), the elimination of the occurrence of post-occlusion recanalization and recurrent reflux following the current thermal or chemical venous ablation. This remains one of the commonest causes of recurrent reflux and varicosities following such treatments. Mechanical occlusion with the AVO, similar to a surgical suture ligation, may also eliminate the need for the extensive length of vessel occlusion necessary to prevent the occurrence of recanalization and recurrence of the reflux in the treated vessels. In those cases where the SFA is too large to provide safe laser or RF endo-ablation partial or complete occlusion of the SFJ with the AVO may be an alternative to open surgery (R).

Using the Amsel Vessel Occluder for targeted vessel occlusion may facilitate the CHIVA method of treating venous reflux with saphenous vein preservation, to be more minimally invasive. With the increasing popularity in the use of chemical agents for vein ablation, prior occlusion of the SFJ and the large tributaries or perforators, to the intravenous injection of foam sclerosants and glues, may be a simple and secure way to prevent or minimize leakage into the deep venous system, avoiding open surgical ligation and minimizing the potential harmful local and systemic effects of these chemicals.

Finally, with such a simple, minimally invasive and reliable interventional method to treat venous reflux, the Amsel Vessel Occluder may allow patients diagnosed with clinically significant reflux and venous insufficiency, prior to the onset of the clinical manifestations of varicose veins, to undergo early intervention to minimize the cosmetic and debilitating consequences of varicose vein and venous insufficiency and their associated health care costs.

The following references are incorporated herein by reference in their entirety:

Gloviczki P, Comerota A J, Dalsing M C, et al: The care of patients with varicose veins and associated chronic venous diseases: Clinical practice guidelines of the Society for Vascular Surgery and the American Venous Forum. J Vasc Surg 2011; 53:2S-48S

Smith J J, Garratt A M, Guest M, Greenhalgh R M, Davies A H. Evaluating and improving health-related quality of life in patients withvaricose veins. J Vasc Surg 1999; 30:710-9.

Korn P, Patel S T, Heller J A, Deitch J S, Krishnasastry K V, Bush H L, et al. Why insurers should reimburse for compression stockings in patients with chronic venous stasis. J Vasc Surg 2002; 35:950-7.

Trendelenburg, F. “Über die Unterbindung der Vena saphena magna bei Unterschenkelvaricen. Brun's] Beitrage zur klinischen Chirurgie 1891, 7: 195-210.

Franceschi C: Theory and Practice of the Conservative Haemodynamic Cure of Incompetent and Varicose Veins in Ambulatory Patients, translated by Evans J. Precy-sous-Thil. 1988

Zamboni P, Escribano J M: Regarding reflux elimination without any ablation or disconnection of the saphenous vein. A haemodynamic model for surgery and ‘durability of reflux-elimination by a minimal invasive CHIVA procedure on patients with varicose veins. a 3-year prospective case study. Eur J Vasc Endovasc Surg 28:567-568, 2004

Miller A, Lilach N, Miller R: A Novel Secure Vessel Occluder for Minimally Invasive and Percutaneous Treatments. J Vasc Surg 2014; 59 (Supp165):PS234-89S.

Miller A, Lilach N, Botero-Anug A, Willenz U, and Miller R: Comparison of a Novel Secure Transfixing Blood Vessel Occluder with the Hemoclip in the Porcine Model. J Laparoendoscopic Surgery. Submitted for publication.

O'Donnell T. F., Balk E. M., Dermody, Tangney E, Iafrati M. D.: Recurrence of varicose veins after endovenous ablation of the great saphenous vein in randomized trials. J Vasc Surg Venous & Lymphatic Disorders 2016; 4, 97-105.

Bush R. G., Bush P., Flanagan J. et al. Factors Associated with Recurrence of Varicose Veins after Thermal Ablation: Results of The Recurrent Veins after Thermal Ablation Study. The Scientific World Journal2014: Article ID 505843, 7 pages

Rabe E, Pannier F. Epidemiology of chronic venous disorders. In: Gloviczki P, editor. Handbook of venous disorders: guidelines of the American Venous Forum. 3rd ed. London: Hodder Arnold; 2009, p.105-10.

Bergan J J, Schmid-Schönbein G W, Coleridge Smith D M, NIcolaides M S, Boisseau M R and Eklof B: Chronic Venous Disease. N Engl J Med 2006; 355:488-98.

### TABLES

TABLE 1
Results Of 30 Percutaneous Occlusion

| | | | | | Confirmed |
|---|---|---|---|---|---|
| | Size | | | Number of AVO | (*U/S and |
| Vessels Occluded | 2-3.5 mms | 3.6-7 mms | >7 mms | Clips/Occlusion | Surgery) |
| Proximal Femoral | | | | | |
| Artery | 0 | 10 | | 1 | Y |
| Vein | 0 | 8 | | 1 (7) | Y |
| | | | | 2** (1) | |
| Distal Femoral | | | | | |
| Artery | 5 | | | 1 | Y |
| Vein | 1 | | | 1 | Y |
| Carotid Artery | | 3 | 1 (8.3 mms) | 1 (2 AVO); 2 (1 AVO) | Y |
| External Jugular Vein | | | 1 (12.7 mms) | 2 | Y |
| Tributary of EJV | | | 1 (9.5 mms) | | |

2 AVO clips placed as1st clip delivered in posterior muscle

TABLE 2
Possible Indications for Amsel Vessel Occluder in the Treatment of Venous Reflux

| | | Adjunctive | Primary | |
|---|---|---|---|---|
| Indication | Site | Procedures | Procedure | Comment |
| Great | Groin | Chemical/Thermal | | High ligation to |
| Saphenous | | Ablation | | prevent thermal or |
| Vein Occlusion | | (Adjunctive) | | chemical injury |
| | | | | (reflux) in femoral |
| | | | | vein |
| Great | Groin, thigh, | CHIVA | | Occlude appropriate |
| Saphenous | leg | | | veins to reduce |
| Vein Occlusion + | | | | superficial |
| tributaries | | | | hypertension as |
| | | | | determined by |
| | | | | ultrasound |
| Tributaries | Groin, thigh, | Chemical/Thermal | | Particularly in short |
| | leg | Ablation | | segment or |
| | | (Adjunctive) | | inaccessible recurrent |
| | | | | veins |
| Recurrences | Groin, thigh, | | AVO | Particularly in short |
| following | leg | | | segment or |
| Chemical and | | | | inaccessible recurrent |
| Thermal | | | | veins |
| Ablation | | | | |
| Great | Groin, thigh, | | AVO | Primary AVO |
| Saphenous | leg | | | treatment single or |
| Vein Occlusion | | | | multiple clips |
| (primary AVO) | | | | |
| Small | High Ligation | | AVO | Minimize nerve |
| Saphenous | | | | injury |
| Perforators | Groin, thigh, | | AVO | Simpler and safer |
| | leg | | | than current endo- |
| | | | | venous/chemical |
| | | | | treatments |

What is claimed is:

1. A device for delivering multiple occluders, the device comprising:
a barrel comprising a first chamber containing an occluder disposed therein, the occluder comprising a distal occlusion element and a proximal occlusion element; and a second chamber substantially similar to the first chamber; and
a pushing member configured to extend axially through the first chamber to deploy the occluder through the distal end of the chamber.

2. The device of claim 1, wherein each chamber comprises a lumen disposed longitudinally within the barrel, and wherein the chambers are parallel to each other.

3. The device of claim 1, wherein the distal occlusion element is separate from, but connectable to, the proximal occlusion element.

4. The device of claim 1, wherein each chamber contains a plurality of occluders disposed serially.

5. The device of claim 1, wherein the distal occlusion element comprises a connecting rod comprising a first connection element; and wherein the proximal occlusion element comprises a second connection element; and wherein when the distal and proximal occlusion elements are pushed together, the connection elements lock together.

6. The device of claim 1, wherein the distal occlusion element is configured to assume a diametrically-reduced configuration when disposed within the chamber and a diametrically-expanded configuration when pushed out of the chamber; and wherein the proximal occlusion element is configured to assume a diametrically-reduced configuration when disposed within the chamber and a diametrically-expanded configuration when pushed out of the chamber.

7. The device of claim 1, wherein the device is configured to deliver occluders laparoscopically.

8. The device of claim 1, wherein each chamber further contains a delivery tube having a proximal end and a distal end; and wherein the occluder is disposed within the delivery tube.

9. The device of claim 8, wherein the delivery tube is a needle configured to deliver the occluders percutaneously.

10. The device of claim 8, further comprising a locking mechanism configured to hold the distal occlusion element to the device after the distal occlusion element has been pushed out of the delivery tube.

11. The device of claim 10, wherein the locking mechanism comprises:
a locking sheath comprising an expandable region; and
a locking rod extending through the locking sheath, the locking rod comprising a locking tip;
wherein when the locking tip is pulled back with respect to the locking sheath, it causes the expandable region to expand.

12. The device of claim 1, wherein each occlusion element comprises a plurality of fingers configured to assume a generally linear shape when in the diametrically-reduced configuration and configured to expand away from each other when in the diametrically-expanded configuration.

13. A device for percutaneously delivering multiple occluders to a hollow structure, the device comprising:
a hollow tube having a proximal end and a distal end;
a plurality of occluders disposed serially within the tube, each occluder comprising a distal occluder element and a proximal occluder element; and
a pushing member configured to push an occluder out of the distal end of the tube.

14. The device of claim 13, wherein the distal occluder element and the proximal occluder element are separate from but connectable to each other.

15. The device of claim 14, wherein the distal occluder element comprises a connecting rod comprising a first connection element; and wherein the proximal occluder element comprises a second connection element; and wherein when the distal and proximal occluder elements are pushed together, the connection elements lock together.

16. The device of claim 13, wherein the distal occluder element is configured to assume a diametrically-reduced configuration when disposed within the tube and a diametrically-expanded configuration when pushed out of the tube; and wherein the proximal occluder element is configured to assume a diametrically-reduced configuration when disposed within the tube and a diametrically-expanded configuration when pushed out of the tube.

17. The device of claim 13, further comprising a locking mechanism configured to hold the distal occluder element to the delivery device after the distal occluder element has been pushed out of the tube.

18. The device of claim 17, wherein the locking mechanism comprises:
a locking sheath comprising an expandable region; and
a locking rod extending through the locking sheath, the locking rod comprising a locking tip;
wherein when the locking tip is pulled back with respect to the locking sheath, it causes the expandable region to expand.

19. The device of claim 13 wherein the tube is a needle configured to deliver the occluders percutaneously.

20. The device of claim 13, wherein each occluder element comprises a plurality of fingers configured to assume a generally linear shape when in the diametrically-reduced configuration and configured to expand away from each other when in the diametrically-expanded configuration.

21. A device for delivering multiple occluders, the device comprising:
a chamber containing a first occluder and a second occluder; and
a pushing member configured to extend axially through the chamber to deploy the occluders through a distal end of the chamber.

22. The device of claim 21, wherein each occluder comprises a distal occlusion element and a proximal occlusion element.

23. The device of claim 21, wherein the chamber comprises an elongated tube.

24. The device of claim 21, wherein the occluders are disposed within the chamber in series.

25. The device of claim 21, further comprising a second chamber, substantially similar to the first chamber, situated parallel to the first chamber.

26. The device of claim 25, wherein the first and second chambers are disposed within a barrel.
